SEQUENCE LISTING (32 sequences)

SEQ ID NO: 1 - DNA, 2157 bp, H. Sapiens
gctcccagccaagaacctcggggccgctgcgcggtggggaggagttccccgaaacccggccgctaagcgaggcctcctcctcccgcagatccgaacggcctgggcggggtcaccccggctgggacaagaagccgccgcctgcctgcccgggcccggggagggggctggggctggggccggaggcggggtgtgagtgggtgtgtgcggggggcggaggcttgatgcaatcccgataagaaatgctcgggtgtcttgggcacctacccgtggggcccgtaaggcgctactatataaggctgccggcccggagccgccgcgccgtcagagcaggagcgctgcgtccaggatctagggccacgaccatcccaacccggcactcacagccccgcagcgcatcccggtcgccgcccagcctcccgcacccccatcgccggagctgcgccgagagccccagggaggtgccatgcggagcgggtgtgtggtggtccacgtatggatcctggccggcctctggctggccgtggccgggcgccccctcgccttctcggacgcggggccccacgtgcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaagtaactgagaccatgcccgggcctcttcactgctgccaggggctgtggtacctgcagcgtgggggacgtgcttctacaagaacagtcctgagtccacgttctgtttagctttaggaagaaacatctagaagttgtacatattcagagttttccattggcagtgccagtttctagccaatagacttgtctgatcataacattgtaagcctgtagcttgcccagctgctgcctgggcccccattctgctccctcgaggttgctggacaagctgctgcactgtctcagttctgcttgaatacctccatcgatggggaactcacttcctttggaaaaattcttatgtcaagctgaaattctctaattttttctcatcacttccccaggagcagccagaagacaggcagtagttttaatttcaggaacaggtgatccactctgtaaaacagcaggtaaatttcactcaaccccatgtgggaattgatctatatctctacttccagggaccatttgcccttcccaaatccctccaggccagaactgactggagcaggcatggcccaccaggcttcaggagtaggggaagcctggagccccactccagccctgggacaacttgagaattccccctgaggccagttctgtcatggatgctgtcctgagaataacttgctgtcccggtgtcacctgcttccatctcccagcccaccagccctctgcccacctcacatgcctccccatggattggggcctcccaggccccccaccttatgtcaacctgcacttcttgttcaaaaatcaggaaaagaaaagatttgaagaccccaagtcttgtcaataacttgctgtgtggaagcagcgggggaagacctagaaccctttccccagcacttggttttccaacatgatatttatgagtaatttattttgatatgtacatctcttattttcttacattatttatgcccccaaattatatttatgtatgtaagtgaggtttgttttgtatattaaaatggagtttgtttgtaaaaaaaaaaaaaaaaaaaa

SEQ ID NO: 2 - protein, 216 aa, H. Sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 3 - DNA, 19 bp, Artificial Sequence
  Synthetic Oligonucleotide
gggtatgtttaatctgtta

SEQ ID NO: 4 - DNA, 22 bp, Artificial Sequence
  Synthetic Oligonucleotide
ttgggtatgtttaatctgttat

SEQ ID NO: 5 - DNA, 97 bp, Artificial Sequence
  Synthetic Oligonucleotide
tgctgttgacagtgagcgctgggtatgtttaatctgttattagtgaagccacagatgtaataacagattaaacatacccaatgcctactgcctcgga

SEQ ID NO: 6 - DNA, 19 bp, Artificial Sequence
  Synthetic Oligonucleotide
gcatgtagtcactttataa

SEQ ID NO: 7 - DNA, 22 bp, Artificial Sequence
  Synthetic Oligonucleotide
atgcatgtagtcactttataag

SEQ ID NO: 8 - DNA, 97 bp, Artificial Sequence
  Synthetic Oligonucleotide
tgctgttgacagtgagcgctgcatgtagtcactttataagtagtgaagccacagatgtacttataaagtgactacatgcattgcctactgcctcgga

SEQ ID NO: 9 - DNA, 19 bp, Artificial Sequence
  Synthetic Oligonucleotide
gaataggcattaacacaaa

SEQ ID NO: 10 - DNA, 22 bp, Artificial Sequence
  Synthetic Oligonucleotide
aagaataggcattaacacaaag

SEQ ID NO: 11 - DNA, 45 bp, Artificial Sequence
  Synthetic Oligonucleotide
agatgtactttgtgttaatgcctattctttgcctactgcctcgga

SEQ ID NO: 12 - DNA, 19 bp, Artificial Sequence
  Synthetic Oligonucleotide
catgaaagtctagaaataa

SEQ ID NO: 13 - DNA, 22 bp, Artificial Sequence
  Synthetic Oligonucleotide
aacatgaaagtctagaaataaa

SEQ ID NO: 14 - DNA, 97 bp, Artificial Sequence
  Synthetic Oligonucleotide
tgctgttgacagtgagcgcacatgaaagtctagaaataaatagtgaagccacagatgtatttatttctagactttcatgtttgcctactgcctcgga

SEQ ID NO: 15 - DNA, 19 bp, Artificial Sequence
  Synthetic Oligonucleotide
aggccagtatgatttataa

SEQ ID NO: 16 - DNA, 22 bp, Artificial Sequence
  Synthetic Oligonucleotide
taaggccagtatgatttataaa

SEQ ID NO: 17 - DNA, 97 bp, Artificial Sequence
  Synthetic Oligonucleotide
tgctgttgacagtgagcgcaaggccagtatgatttataaatagtgaagccacagatgtatttataaatcatactggccttatgcctactgcctcgga

SEQ ID NO: 18 - DNA, 21 bp, Artificial Sequence
  Synthetic Oligonucleotide
acatgatatttatgagtaatt

SEQ ID NO: 19 - DNA, 22 bp, Artificial Sequence
  Synthetic Oligonucleotide
aacatgatatttatgagtaatt

SEQ ID NO: 20 - DNA, 97 bp, Artificial Sequence
  Synthetic Oligonucleotide
tgctgttgacagtgagcgcacatgatatttatgagtaatttagtgaagccacagatgtaaattactcataaatatcatgtttgcctactgcctcgga

SEQ ID NO: 21 - DNA, 19 bp, Artificial Sequence
  Synthetic Oligonucleotide
caggtgatccactctgtaa

SEQ ID NO: 22 - DNA, 22 bp, Artificial Sequence
  Synthetic Oligonucleotide
aacaggtgatccactctgtaaa

SEQ ID NO: 23 - DNA, 97 bp, Artificial Sequence
  Synthetic Oligonucleotide
tgctgttgacagtgagcgcacaggtgatccactctgtaaatagtgaagccacagatgtatttacagagtggatcacctgtttgcctactgcctcgga

SEQ ID NO: 24 - DNA, 19 bp, Artificial Sequence
  Synthetic Oligonucleotide
cctcgaggttgctggacaa

SEQ ID NO: 25 - DNA, 22 bp, Artificial Sequence
  Synthetic Oligonucleotide
tccctcgaggttgctggacaag

SEQ ID NO: 26 - DNA, 97 bp, Artificial Sequence
  Synthetic Oligonucleotide
tgctgttgacagtgagcgcccctcgaggttgctggacaagtagtgaagccacagatgtacttgtccagcaacctcgagggatgcctactgcctcgga

SEQ ID NO: 27 - DNA, 19 bp, Artificial Sequence
  Synthetic Oligonucleotide
cacgttctgtttagcttta

SEQ ID NO: 28 - DNA, 22 bp, Artificial Sequence
  Synthetic Oligonucleotide
tccacgttctgtttagctttag

SEQ ID NO: 29 - DNA, 97 bp, Artificial Sequence
  Synthetic Oligonucleotide
tgctgttgacagtgagcgcccacgttctgtttagctttagtagtgaagccacagatgtactaaagctaaacagaacgtggatgcctactgcctcgga

SEQ ID NO: 30 - DNA, 21 bp, Artificial Sequence
  Synthetic Oligonucleotide
agctgaaattctctaattttt

SEQ ID NO: 31 - DNA, 22 bp, Artificial Sequence
  Synthetic Oligonucleotide
aagctgaaattctctaattttt

SEQ ID NO: 32 - DNA, 97 bp, Artificial Sequence
  Synthetic Oligonucleotide
tgctgttgacagtgagcgcagctgaaattctctaattttttagtgaagccacagatgtaaaaaattagagaatttcagctttgcctactgcctcgga

(19) United States
(12) Patent Application Publication
(10) Pub. No.: US 2009/0226459 A1
(43) Pub. Date: Sep. 10, 2009

(54) ROLE OF FGF-19 IN CANCER DIAGNOSIS AND TREATMENT

(75) Inventor: SCOTT POWERS, GREENLAWN, NY (US)

Correspondence Address: WOLF GREENFIELD & SACKS, P.C., 600 ATLANTIC AVENUE, BOSTON, MA, 02210-2206, US

(73) Assignee: COLD SPRING HARBOR LABORATORY, COLD SPRING HARBOR, NY (US)

(21) Appl. No.: 12/362,473

(22) Filed: Jan. 29, 2009

Related U.S. Application Data
(60) Provisional application No. 61/062,952, filed on Jan. 29, 2008.

(51) Int. Cl. C12Q 1/68; C40B 30/04; G01N 33/53; A61K 39/395

(52) U.S. Cl. 424/145.1; 435/6; 506/9; 435/7.1; 435/7.92

(57) ABSTRACT

The present invention relates, in part, to the discovery that human FGF19 is amplified in a number of cancers, including liver and esophageal cancers, and that this amplification correlates with over-expression of this gene. In some aspects, the invention provides methods and kits for diagnosing a patient having or at risk for developing cancer, such as liver or esophageal cancer. The invention in other aspects provides methods for selecting a treatment for a patient having cancer. In other aspects, the invention relates to methods for treating cancer using an FGF19 inhibitor.

### RELATED APPLICATION

This application claims priority under 35 U.S.C. §119(e) from U.S. provisional application Ser. No. 61/062,952 filed Jan. 29, 2008, the entire contents of which are incorporated by reference herein in their entirety.

### FUNDING

This invention was made with United States government support under grant 1 R01 CA1246848-01, awarded by the National Institutes of Health. The United States government has certain rights in the invention.

### FIELD OF THE INVENTION

The present invention relates to the field of cancer therapy and methods for selecting treatments for certain cancers.

### BACKGROUND OF THE INVENTION

Fibroblast growth factor 19 (FGF-19) is a member of the fibroblast growth factor (FGF) family of secreted growth factors. The FGF family is a large group of growth factors found in organisms as diverse as C. elegans and H. sapiens (Itoh et al., 2004, Trends Genet. 20:563-9). In vertebrates, there are thus far twenty-two known members of this family that differentially activate four distinct FGF receptors. The FGF family members play distinct roles in the regulation of proliferation, migration, and differentiation during embryonic development (Ornitz et al., 2001, Genome Biol 2:REVIEWS 3005).

FGF19 is unique amongst the FGF molecules in that it has affinity for only one FGF receptor, FGFR4 (Xie et al., 1999, Cytokine 11:729-35). FGF19 expression has been observed in a wide range of fetal tissues and in the adult gall bladder (Nishimura et al., 1999, Biochim. Biophys. Acta 1444:148-51; Xie et al., 1999, Cytokine 11:729-35). Analysis of chicken embryos has shown that FGF19 is a key regulator of the development of the inner ear (Ladher et al., 2000, Science 290:1965-7). Ectopic expression of FGF19 can also cause weight loss in mice (Yu et al., 2002, Am. J. Pathol. 161:2003-10; Yu et al., 2000, J. Biol. Chem. 275:15482-9).

In adult tissues, FGF molecules function in injury and tissue repair, and many of them are over-expressed in cancer cell lines and can malignantly transform 3T3 cells when over-expressed (Ornitz et al., supra). In humans, three of the four genes for FGF receptors undergo mutational activation in different forms of cancer (Cappellen et al., 1999, Nat Genet. 23:18-20; Richelda et al., 1997, Blood 90:4062-70; Xiao et al., 1998, Nat Genet. 18:84-7).

Certain primary cancers (e.g., liver and esophageal cancers) are difficult to discover early and often do not respond to current treatments. The prognosis is often poor. Accordingly, there is a need for diagnostic methods that can identify individuals that may be suffering from such cancers, or are at risk for developing such cancers during their lifetimes. Diagnostic methods would also be useful in screening individuals for potential treatments with anti-cancer therapies.

### SUMMARY OF THE INVENTION

The present invention is based on the discovery that human FGF19 is amplified in a number of cancers, including liver and esophageal cancers, and that this amplification correlates with over-expression of this gene in cancer. The present invention is also based on the surprising discovery that amplification and over-expression of FGF19 is functionally important in cancer cells, and not just a passive result of the nearby oncogene CCND1, as was previously thought. Accordingly, the invention provides methods for selecting a patient for treatment with an inhibitor of FGF19, and for treating a patient with liver or esophageal cancer. The invention also provides methods for diagnosing a patient having or at risk for developing cancer, such as liver or esophageal cancer. The present invention is also based on the discovery that in certain cancers amplification of FGF19, alone, is not indicative of a cancer that is responsive to treatment with an FGF19 inhibitor (an inhibitor of FGF19) and that over-expression FGF19 (protein, mRNA) and amplification of FGF19 (e.g., amplification of the 11q13 locus) is required. Accordingly, the invention provides methods for diagnosing cancer, and methods for selecting a treatment for a patient having cancer, based on amplification of certain genomic sequences that are indicative of amplification of the FGF19 gene (e.g., 11q13, 11q13.3, FGF19 gene) and over-expression of the FGF19 gene (e.g., protein, mRNA).

The invention in some aspects relates to methods of identifying a patient as a candidate for treatment with an FGF19 inhibitor. In some embodiments, the methods involve obtaining a clinical sample from a patient having cancer, determining if a 11q13 locus (e.g., 11q13.3) is amplified in the clinical sample, and determining if FGF19 is over-expressed in the clinical sample, and, if the clinical sample has amplification of the 11q13 locus and over-expression of FGF19, identifying the patient as a candidate for treatment with an FGF19 inhibitor. In some embodiments, methods involve obtaining a clinical sample that is suspected of having an amplification of a 11q13 locus from a patient having cancer, determining if FGF19 is over-expressed in the clinical sample, and, if the clinical sample has over-expression of FGF19, identifying the patient as a candidate for treatment with an FGF19 inhibitor. In certain embodiments, the cancer is liver cancer, esophageal cancer or stomach cancer. In some embodiments, methods involve obtaining a clinical sample from a patient having liver, esophageal, stomach, or related cancer; determining if a 11q13 locus is amplified in the clinical sample, and, if the clinical sample has amplification of the 11q13 locus, identifying the patient as a candidate for treatment with an FGF19 inhibitor.

In certain embodiments, the step of determining if a 11q13 locus is amplified comprises measuring copy number of a 11q13 locus in the clinical sample. In certain embodiments, the step of determining if a 11q13 locus is amplified comprises comparing a copy number of the 11q13 locus in the clinical sample with a control sample, wherein an increase in copy number of the 11q13 locus compared with the control sample indicates that the 11q13 locus is amplified. The control sample may be a cell or tissue sample (e.g., of the same cell or tissue type as the cancer sample) that is normal (e.g., cancer-free from a healthy subject).

In certain embodiments, the step of determining if a 11q13 locus is amplified comprises measuring the copy number of FGF19 in the clinical sample. In certain embodiments, the step of determining if a 11q13 locus is amplified comprises comparing a copy number of FGF19 in the clinical sample with a control sample, wherein an increase in copy number of FGF19 compared with the control sample indicates that the 11q13 locus is amplified.

In certain embodiments, the step of determining if a 11q13 locus is amplified comprises combining the clinical sample with a polynucleotide probe that hybridizes, under stringent conditions, to the 11q13 locus; detecting hybridization of the polynucleotide probe; and comparing the amount of hybridization that occurs in the clinical sample to the amount of hybridization that occurs in a control sample comprising a reference tissue, wherein an increased level of hybridization in the clinical sample relative to the control sample indicates that the 11q13 locus is amplified. In specific embodiments, the polynucleotide probe comprises a sequence complementary to a FGF19 genomic sequence. In specific embodiments, the polynucleotide probe is a FISH probe, a Southern blot probe, a real-time PCR probe, array probe or a bead array probe.

In certain embodiments, the step of determining if a 11q13 locus is amplified comprises combining the clinical sample with a first polynucleotide probe that hybridizes, under stringent conditions, to the 11q13 locus; combining the clinical sample with a second polynucleotide probe that hybridizes, under stringent conditions, adjacent to the first probe; ligating the first and second polynucleotide probes to form a target probe; amplifying the target probe; and detecting the amount of amplified target probe, wherein an increased level of target probe in the clinical sample relative to a control sample comprising a reference tissue indicates that the 11q13 locus is amplified. In specific embodiments, the target probe comprises a sequence complementary to a FGF19 genomic sequence.

In certain embodiments, the step of determining if a 11q13 locus is amplified comprises combining the clinical sample with a pair of polynucleotide primers that hybridize, under stringent conditions, to the 11q13 locus; amplifying DNA in the sample, thereby producing amplified DNA; and detecting the amount of amplified DNA, wherein an increased level of amplified DNA in the clinical sample relative to a control sample comprising a reference tissue indicates that the 11q13 locus is amplified. In specific embodiments, the polynucleotide primers comprise a sequence complementary to a FGF19 genomic sequence.

In some embodiments, a marker for the 11q13.3 genomic region (the genomic region that comprises the FGF19 gene) may be used. In some embodiments, the marker may be used to detect amplification (e.g., an above-normal copy number of) the FGF19 genetic locus, the Cyclin D1 genetic locus, an adjacent genetic locus, the 11q13.3 locus or any combination thereof. In some embodiments, the genetic locus may be used using one or more oligonucleotides (e.g., in a hybridization, PCR, LCR, or other suitable assay). In some embodiments, a BAC comprising one or more genomic sequences from the 11q13 region (e.g., the 11q13.3 region) may be used to detect amplification according to methods of the invention. For example, one or more of the non-limiting BAC constructs illustrated in FIG. 15 may be used.

In some embodiments, the step of determining if FGF19 is over-expressed comprises measuring expression of FGF19 mRNA in the clinical sample. In some embodiments, the step of determining if FGF19 is over-expressed comprises comparing expression of FGF19 mRNA in the clinical sample with a control sample, wherein an increase in expression of FGF19 mRNA in the clinical sample compared with the control sample indicates that FGF19 is over-expressed. The control sample may be a cell or tissue sample (e.g., of the same cell or tissue type as the cancer sample) that is normal (e.g., cancer-free from a healthy subject).

In certain embodiments, the measuring comprises performing real-time PCR, FISH, northern analysis, a RNAse protection assay, microarray analysis, or bead array analysis to detect FGF19 mRNA.

In some embodiments, the step of determining if FGF19 is over-expressed comprises measuring expression of FGF19 protein in the clinical sample. In some embodiments, the step of determining if FGF19 is over-expressed comprises comparing expression of FGF19 protein in the clinical sample with a control sample, wherein an increase in expression of FGF19 protein in the clinical sample compared with the control sample indicates that FGF19 is over-expressed. In specific embodiments, the measuring comprises performing an ELISA or Immunohistochemistry to detect FGF19 protein.

In some embodiments, the clinical sample is a tissue biopsy. In certain embodiments, the tissue is a liver, esophageal, or stomach tissue.

In some embodiments, the FGF19 inhibitor is selected from the group consisting of: an anti-FGF19 antibody or an antigen-binding fragment thereof, an anti-FGF19 antisense molecule, and, an aptamer, siRNA or miRNA against FGF19. In certain embodiments, the anti-FGF19 antibody is a mouse anti-human FGF-19 monoclonal antibody 1A6 or an antibody comprising a variable region from a mouse anti-human FGF-19 monoclonal antibody 1A6. In certain embodiments, the FGF19 inhibitor is a siRNA or shRNA against FGF19 comprising a sequence selected from the sequences set forth in SEQ ID NO: 18-32.

According to some aspects of the invention, methods are provided for treating a patient having liver, esophageal, or stomach cancer, wherein amplification of the 11q13 locus (e.g., 11q13.3) is detected in a clinical sample from the patient. In some embodiments, methods involve administering to the patient an effective amount of an FGF19 inhibitor. According to other aspects of the invention, methods are provided for treating a patient having cancer, wherein amplification of the 11q13 locus and over-expression of FGF19 is detected in a clinical sample from the patient. In some embodiments, methods administering to the patient an effective amount of an FGF19 inhibitor. In some embodiments, the FGF19 inhibitor is selected from the group consisting of: an anti-FGF19 antibody or an antigen-binding fragment thereof, an anti-FGF19 antisense molecule, and, an aptamer, siRNA, shRNA or miRNA against FGF19. In some embodiments, the FGF19 inhibitor is a siRNA or shRNA against FGF19 comprising a sequence selected from the sequences set forth in SEQ ID NO: 18-32.

According to some aspects of the invention, kits are provided for diagnosing cancer in a patient. In some embodiments, the kits comprise at least one container having disposed therein a polynucleotide probe that hybridizes, under stringent conditions, to the 11q13 locus, at least one container having disposed therein a reagent for detecting expression of the FGF19 gene, and a label and/or instructions for the use of the diagnostic kit in the detection of amplification of the 11q13 locus and expression of the FGF19 gene in the sample.

In one embodiment, the present invention provides a method of selecting a patient for treatment with an inhibitor of FGF19. Such a method comprises providing a DNA sample from the patient and detecting, in the DNA sample, amplification of a FGF19 gene or portion thereof. Amplification indicates that the patient is a candidate for said treatment. In one embodiment, the detecting step comprises combining the sample with a polynucleotide probe that hybridizes, under stringent conditions, to the FGF19 gene or portion, detecting hybridization of the polynucleotide probe, and comparing the amount of hybridization that occurs in the sample to the amount of hybridization that occurs in a control sample comprising a reference tissue, wherein an increased level of hybridization in the patient DNA sample relative to the control sample indicates that the patient has or is at risk for developing cancer.

In another embodiment, the detecting step comprises: i) combining the sample with a first polynucleotide probe that hybridizes, under stringent conditions, to the FGF19 gene or portion, ii) combining the sample with a second polynucleotide probe that hybridizes, under stringent conditions, adjacent to the first probe, iii) ligating the first and second polynucleotide probes to form a target probe, iv) amplifying the target probe, and, iv) detecting the amount of amplified target probe, wherein an increased level of target probe in the patient DNA sample relative to a control sample comprising a reference tissue indicates that the patient is a candidate for said treatment. In a specific embodiment, the detecting step of the method comprises ligation-dependent probe amplification to determine whether the FGF19 gene or portion is amplified.

In another embodiment, the detecting step comprises: i) combining the sample with a pair of polynucleotide primers that hybridize, under stringent conditions, to the FGF19 gene or portion, ii) amplifying DNA in the sample, thereby producing amplified DNA, and, iii) detecting the amount of amplified DNA, wherein an increased level of amplified DNA in the patient DNA sample relative to a control sample comprising a reference tissue indicates that the patient is a candidate for said treatment. In a specific embodiment, the detecting step of the method comprises real-time PCR to determine whether the FGF19 gene or portion is amplified.

As used herein, a reference tissue can be from a normal individual (e.g., an individual not having or not at risk for developing cancer) or from a normal tissue (e.g., not cancerous) of the patient.

In another embodiment, the detecting step of the method comprises sequencing a region of the DNA that comprises the FGF19 gene or portion and determining whether the FGF19 gene or portion is amplified.

As used herein, the term “FGF19”, unless otherwise indicated, refers to, FGF19 protein, FGF19 mRNA, or a genomic sequence of the FGF19 gene (a FGF19 genomic sequence). Several exemplary FGF19 genomic, mRNA, and protein sequences are disclosed herein. Other FGF19 sequences will be apparent to the skilled artisan.

As used herein, the term “FGF19 gene” refers to the genomic locus that determines expression of the FGF19 gene product which comprises regulatory (e.g., promoters, enhancers, silencers), coding, and non-coding (e.g., intronic sequence) nucleotide sequences.

As used herein, the term “portion” refers to all or any amount up to all of the object. For example, a portion of the FGF19 gene can comprise all of the nucleotides that comprise the FGF19 gene, one of the nucleotides that comprises the FGF19 gene, or any number (e.g., 2, 3, 4, 5, 10, 20, 25, 50, 100, 200, 300, 400, 500, 1000, 1500, or more than 1500) of the nucleotides that comprise the FGF19 gene. As used herein, “increased level” refers to any level of increase as compared to a reference level (e.g., the level in a control sample, a basal state level, or a level found in a different condition or time). For example, an increased level can be any level greater than one-fold, such as 1.1-fold, 1.25-fold, 1.5-fold, 2-fold, 2.25-fold, 2.5-fold, 3-fold, 3.5-fold, 4-fold, 5-fold, 6-fold, 7-fold, 8-fold, 9-fold, 10-fold, 15-fold, 20-fold, 25-fold, or greater than 25-fold.

As used herein, a “clinical sample” refers to an isolated biomolecule, such as DNA, RNA, or protein, an isolated cell, an isolated tissue, saliva, gingival secretions, cerebrospinal fluid (spinal fluid), gastrointestinal fluid, mucus, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, stool, and nasal secretions. In some instances, a clinical sample is a tissue biopsy, such as a tumor or cancer biopsy. However, clinical samples are not so limited and other exemplary clinical samples will be readily apparent to one of ordinary skill in the art.

As used herein, “obtaining a clinical sample” refers to any process for directly or indirectly acquiring a clinical sample from a patient. For example, a clinical sample may be obtained (e.g., at a point-of-care facility) by procuring a tissue sample (e.g., a liver or esophageal tissue sample) from a patient or procuring a specimen, such as a urine sample, produced by the patient. Alternatively, a clinical sample may be obtained by receiving the clinical sample (e.g., at a laboratory facility) from one or more individuals who procured the sample from the patient.

As used herein the phrase “determining if a 11q13 locus is amplified” refers to the process of measuring the amount of 11q13 loci in a clinical sample. The determining process may include any number of steps necessary for evaluating 11q13 levels in a clinical sample. For example, determining may involve preparing a cell, chromosome (e.g., mitotic spread) or DNA sample from a clinical sample. The step of determining may include any one of a number of biochemical steps associated with preparing reagents (e.g., polynucleotide probes, primers, etc.) useful for detecting 11q13 levels. The step of determining may include the process of labeling a clinical sample (e.g., hybridizing polynucleotide probes and primers to nucleic acids of the clinical sample) and measuring 11q13 levels (e.g., detecting fluorescent labels, e.g., qPCR probes or reagents, FISH probes, etc.). The step of determining may also involve quantifying 11q13 levels (e.g., quantifying levels of a FGF19 genomic sequence). For example, the amount of 11q13 may be evaluated as a number of copies (e.g., average number of copies) of the 11q13 locus per cell. The step of determining may also involve comparing the 11q13 levels in the clinical sample to a control sample (e.g., a reference level representing 11q13 levels in a reference tissue). In a case where a control sample represents 11q13 levels in a normal tissue, 11q13 levels in a clinical sample that are higher than levels in the control sample are indicative of amplification of the 11q13 locus in the clinical sample. Other steps associated with determining if a 11q13 locus is amplified are disclosed herein. These, as well as others, will be apparent to the skilled artisan.

As used herein, the phrase “determining if FGF19 is over-expressed” refers to the process of measuring the amount of FGF19 expression in a clinical sample. The determining process may include any number of steps necessary for evaluating FGF19 expression (e.g., mRNA or protein expression) in a clinical sample. For example, determining may involve preparing a cell, tissue section, RNA, or protein sample from a clinical sample. The step of determining may include any one of a number of biochemical steps associated with preparing reagents (e.g., polynucleotide probes, primers, antibodies, etc.) useful for detecting FGF19 expression, e.g., mRNA or protein. The step of determining may include the process of labeling a clinical sample (e.g., hybridizing polynucleotide probes and primers to nucleic acids of the clinical sample, e.g., for detecting mRNA, labeling the clinical sample with an antibody, e.g., for immunohistochemistry) and measuring FGF19 expression levels (e.g., detecting fluorescent labels, e.g., qRT-PCR probes or reagents, detecting antibody labels, e.g., enzymatic labeling, e.g., alkaline phosphates, etc.). The step of determining may also involve quantifying FGF19 expression levels (e.g., quantifying levels of the FGF19 expression). For example, the amount of FGF19 may be evaluated relative to total protein levels or total mRNA levels, as appropriate. The step of determining may also involve comparing FGF19 expression levels in the clinical sample to a control sample (e.g., a reference level representing FGF19 expression levels in a reference tissue). In a case where a control sample represents FGF19 expression levels in a normal tissue, FGF19 expression levels in a clinical sample that are higher than levels in the control sample are indicative of FGF19 over-expression in the clinical sample. Other steps associated with determining if FGF19 is over-expressed are disclosed herein. These, as well as others, will be apparent to the skilled artisan.

As used herein, the term “reference tissue” refers to a non-diseased (normal) tissue. A reference tissue may be obtained, for example, from a patient suspected of being disease-free or having non-diseased (normal) tissue.

As used herein, the phrase “having cancer” refers to a patient having or suspected of having cancer.

In certain embodiments, the method is used to select a patient having or at risk for developing liver cancer or esophageal cancer.

In one embodiment, the inhibitor of FGF19 is selected from the group consisting of an anti-FGF19 antibody or an antigen-binding fragment thereof, an anti-FGF19 antisense molecule, and, an aptamer, small interfering RNA (siRNA) or microRNA (miRNA) against FGF19.

In another embodiment, the present invention provides a method of diagnosing a patient having or at risk for developing cancer. Such a method comprises providing a DNA sample from the patient and detecting, in the DNA sample, amplification of a FGF19 gene or portion. Amplification of the FGF19 gene or portion indicates that the patient has or is at risk for developing cancer.

In one embodiment, the detecting step in such a method comprises combining the sample with a polynucleotide probe that hybridizes, under stringent conditions, to the FGF19 gene or portion, detecting hybridization of the polynucleotide probe, and comparing the amount of hybridization that occurs in the sample to the amount of hybridization that occurs in a control sample comprising a reference tissue, wherein an increased level of hybridization in the patient DNA sample relative to the control sample indicates that the patient has or is at risk for developing cancer.

In another embodiment, the detecting step comprises: i) combining the sample with a first polynucleotide probe that hybridizes, under stringent conditions, to the FGF19 gene or portion, ii) combining the sample with a second polynucleotide probe that hybridizes, under stringent conditions, adjacent to the first probe, iii) ligating the first and second polynucleotide probes to form a target probe, iv) amplifying the target probe, and, iv) detecting the amount of amplified target probe, wherein an increased level of target probe in the patient DNA sample relative to a control sample comprising a reference tissue indicates that the patient has or is at risk for developing cancer. In a specific embodiment, the detecting step of the method comprises ligation-dependent probe amplification to determine whether the FGF19 gene or portion is amplified.

In another embodiment, the detecting step comprises: i) combining the sample with a pair of polynucleotide primers that hybridize, under stringent conditions, to the FGF19 gene or portion, ii) amplifying DNA in the sample, thereby producing amplified DNA, and, iii) detecting the amount of amplified DNA, wherein an increased level of amplified DNA in the patient DNA sample relative to a control sample comprising a reference tissue indicates that the patient has or is at risk for developing cancer. In a specific embodiment, the detecting step of the method comprises real-time PCR to determine whether the FGF19 gene or portion is amplified.

A reference tissue can be from a normal individual (e.g., an individual not having or not at risk for developing cancer) or from a normal tissue (e.g., not cancerous) of the patient.

In another embodiment, the detecting step of the method comprises sequencing a region of the DNA that comprises the FGF19 gene or portion and determining whether the FGF19 gene or portion is amplified.

In certain embodiments, the method is used to select a patient having or at risk for developing liver cancer or esophageal cancer.

In one embodiment, the inhibitor of FGF19 is selected from the group consisting of an anti-FGF19 antibody or an antigen-binding fragment thereof, an anti-FGF19 antisense molecule, and, an aptamer, siRNA or miRNA against FGF19.

Generally, DNA samples used in the invention are taken from cells or tissue from the liver or esophagus of a patient. However, in certain embodiments, DNA samples may be taken from cells or tissue of, for example, the stomach, colon, lung, breast, eye, ear, throat, nose, tongue, epidermis, epithelium, blood, saliva, mucus, urinary tract, bladder, kidney, prostate, urine, muscle, bone, cartilage, or skin.

The polynucleotides described herein (e.g., a polynucleotide probe or a polynucleotide primer) may be DNA or RNA. The subject polynucleotide may be single-stranded or double-stranded. Polynucleotide probes and primers of the invention may be from about 5 nucleotides to about 3000 nucleotides. In some embodiments, the polynucleotide probes and primers of the invention are from about 8 nucleotides to about 500 nucleotides. In other embodiments, the polynucleotide probes and primers of the invention are from about 10 nucleotides to about 250 nucleotides. In certain embodiments, the subject polynucleotide probes and primers are about 20 nucleotides (e.g., 15, 16, 17, 18, 19, 20, 21, 22, 23, 24, 25, 26, 27, 28, 29, or 30 nucleotides). In other embodiments, the subject polynucleotide probes and primers are from about 50 to about 100 nucleotides (e.g., 45, 50, 55, 60, 65, 75, 85, or 100 nucleotides). The subject polynucleotides may comprise one or more non-natural or modified nucleotides. Non-natural or modified nucleotides include, without limitation, radioactively, fluorescently, or chemically labeled nucleotides.

In another embodiment, the present invention provides a method of treating a patient with liver or esophageal cancer, wherein amplification of a FGF19 gene is detected in a DNA sample from the patient. Such a method comprises administering to a patient an effective amount of an inhibitor of FGF19. Inhibitors of FGF19 include, without limitation, nucleic acids such as antisense nucleic acids, RNA interference nucleic acids, miRNA nucleic acids, and siRNA nucleic acids. Other inhibitors include antibodies, ribozymes, aptamers, and small molecules. In one embodiment, the inhibitor of FGF19 is selected from the group consisting of an anti-FGF19 antibody or an antigen-binding fragment thereof, an anti-FGF19 antisense molecule, and, an aptamer, siRNA or miRNA against FGF19. In another embodiment, the inhibitor of FGF19 is an RNAi, siRNA, or antisense nucleic acid that inhibits the expression or activity of FGF19.

The invention also provides non-human animals that are useful for understanding cancer and its treatments. These animals are transgenic or chimeric animals at least some of whose liver or esophageal cells over-express FGF19. These animals are susceptible to developing cancer in tissues containing those cells. Those cells can further contain a genetic mutation (e.g., another activated oncogene (ras, Akt, or myc) or a defect in a tumor suppressor gene, like p53 and PTEN) that makes them even more susceptible to cancer.

In one embodiment, the invention provides a mouse at least some of whose liver or esophageal cells comprise a genome comprising a heterologous nucleic acid sequence comprising a FGF19 coding sequence and an expression control sequence linked operatively thereto, wherein the mouse has cancer, or is more susceptible of developing cancer as compared to a control mouse not having said heterologous nucleic acid sequence. In one embodiments, the mouse is a chimeric mouse some or all of whose hepatocytes comprise said genome. In another embodiment, the mouse is a chimeric mouse some or all of whose esophageal cells comprise said genome.

The present invention also provides a method of identifying a molecule useful for treating cancer. Such a method comprises providing a mouse described herein, wherein the mouse has developed cancer, and treating the mouse with a candidate molecule. Inhibition of the growth of said cancer or a regression of said cancer indicates the candidate molecule is useful for treating cancer.

In certain embodiments, the invention provides a diagnostic kit for detecting amplification of a FGF19 gene in a sample from a patient. In one embodiment, the diagnostic kit comprises at least one container means having disposed therein a polynucleotide probe that hybridizes, under stringent conditions, to the FGF19 gene and a label and/or instructions for the use of the diagnostic kit in the detection of amplification of the FGF19 gene in the sample. In another embodiment, the diagnostic kit comprises at least one container means having disposed therein a polynucleotide primer that hybridizes, under stringent conditions, adjacent, upstream, downstream or to the FGF19 gene and a label and/or instructions for the use of the diagnostic kit in the detection of amplification of the FGF19 gene in a sample. The probe or primer of the invention may hybridize to a portion of the FGF19 gene, or may overlap completely with the FGF19 gene. A diagnostic kit of the invention may additionally comprise a second polynucleotide primer that hybridizes, under stringent conditions, to the other side of the FGF19 gene.

In another embodiment, the present invention provides a kit comprising at least one container means comprising a premeasured dose of one or more inhibitors of FGF19 and a label or instructions for use of the kit.

### BRIEF DESCRIPTION OF THE DRAWINGS

FIG. 1A is a graph showing the correlation of DNA copy number and relative expression for CCND1 and FGF19 in liver tumors. Circles ═CCND1 (corr=0.68); Triangles=FGF19 (corr=0.46). FIG. 1B is a graph showing the correlation of DNA copy number and relative expression for CCND1 and FGF19 in esophageal tumors. Circles=CCND1 (corr=0.31); Triangles=FGF19 (corr=0.69).

FIG. 2 is a graph showing TaqMan Analysis of the relationship between 11q13 amplicon DNA copy number and FGF19 Expression in primary breast tumors.

FIG. 3 depicts detection of Fgf19 protein by immunoblotting in human HCC cell lines. In panel A, lane 1 contains protein extract from a mouse cell line (the antibody does not react with the mouse ortholog of FGF19), lane 2 is a colon cancer cell line that expresses large amounts of Fgf19, lane 3: Alex cells, lane 4: Huh7; 5: Hep3B; 6: HepG2, 7: THLE-3; and lane 8: SK-Hep1. In panel B, lanes 1 & 2 as above in Panel A, lane 3: Huh7, lane 4: SNU182, 5: SNU387, 6: SNU398, 7: SNU423, 8: SNU449, 9: SNU475. Panel C depicts the ability of different shRNAs to reduce FGF19 protein levels in a human cancer cell line. Lanes 1 and 6, shows control cells containing empty vector, Lanes 2 through 5, show anti-FGF19 K1, K2, K4, and K5 respectively.

FIG. 4 depicts the ability of different shRNAs to reduce tumor formation in nude mice. Panel A shows tumor formation assays. Ten million Li7 cells harboring different shRNAs were injected into five mice (both flanks), providing 10 sites for tumor formation. At the indicated days, tumor volumes were measured. At the final day, ANOVA analysis indicates statistically significant tumor suppression (* indicates p<0.01; ** indicates p<0.05). Panel B, as above but with 7 million Huh7 cells injected.

FIG. 5 depicts the effects of FGF19 knock-down on proliferation of Huh7 cells.

FIG. 6 depicts the effects of Cyclin D1 knock-down on proliferation of Huh7 cells.

FIG. 7 depicts the effects of FGF19 knock-down on proliferation of HepG2 cells.

FIG. 8 depicts the effects on Cyclin D1 knock-down on proliferation of HepG2 cells.

FIG. 9 depicts the effects of FGF19 knock-down on proliferation of SNU182 cells.

FIG. 10 depicts the effects of Cyclin D1 knock-down on proliferation of SNU182 cells.

FIG. 11 depicts the effects of FGF19 knock-down on proliferation of SNU423 cells.

FIG. 12 depicts the effects of Cyclin D1 knock-down on proliferation of SNU423 cells.

FIG. 13 depicts the effects of FGF19 knock-down on proliferation of Li7 cells.

FIG. 14 depicts the effects on Cyclin D1 knock-down on proliferation of Li7 cells.

FIG. 15 depicts a portion of the 11q13 locus from the UCSC Genome Browser based on the March 2006 assembly. Corresponding BAC clones and UCSC genes are shown.

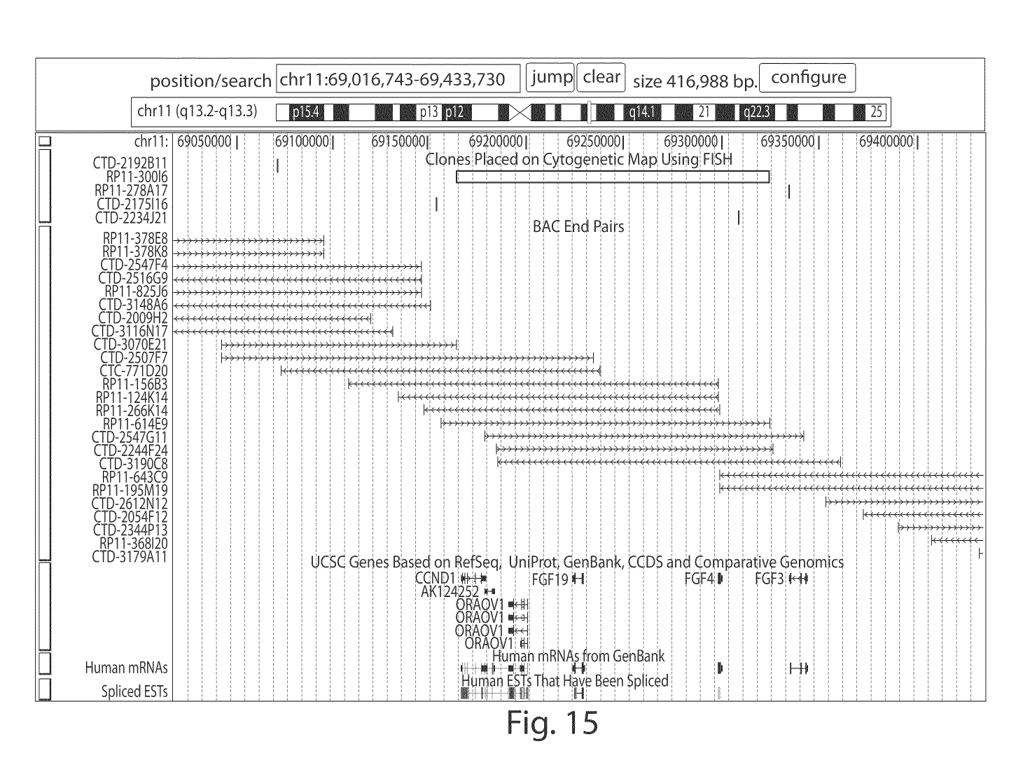

### DETAILED DESCRIPTION OF THE INVENTION

Aspects of the invention relate to methods and compositions for diagnosing and/or treating certain cancers associated with genomic amplification of the 11q13 genomic region. Aspects of the invention are based, at least in part, on the discovery that certain cancers associated with 11q13 amplification involve FGF19 over-expression (e.g., FGF19 over-expression drives cell growth and proliferation in these cancers). Accordingly, certain methods and compositions of the invention involve detecting FGF19 amplification, detecting FGF19 expression, and/or inhibiting FGF19 expression and/or activity. In some embodiments, FGF19 is not over-expressed in certain cancers where the FGF19 gene is amplified. Accordingly, in some embodiments, FGF19 amplification alone is not sufficient for determining whether a cancer is associated with FGF19 activity nor whether it will be responsive to treatment with an FGF19 inhibitor. Accordingly, aspects of the invention relate to using FGF19 expression (e.g., over-expression relative to a non-cancerous sample) as a marker for determining whether a cancer (e.g., a cancer that is associated with 11q13 genomic amplification or a cancer that is associated with FGF19 genomic amplification) is a candidate for treatment with an FGF19 inhibitor. However, in some embodiments, FGF19 gene amplification is associated with FGF19 over-expression in cancers of certain tissues, for example in liver and esophageal cancers. Accordingly, FGF19 amplification alone may be used as a marker for determining whether certain cancers (e.g., liver, esophageal, and related cancers such as stomach cancer) are candidates for treatment with an FGF19 inhibitor.

Accordingly, aspects of the invention are useful for determining whether FGF19 is an important contributor to a particular cancer (e.g., a driver of that cancer). In some embodiments, detecting FGF19 amplification (e.g., an above-normal genomic copy number of FGF19) is not sufficient to determine whether FGF19 is an important contributor to a cancer. In some embodiments, detecting FGF19 over-expression is indicative that FGF19 is a driver of a particular cancer. In some embodiments, detecting both FGF19 genomic amplification and FGF19 over-expression is indicative that FGF19 is a driver of a particular cancer. Accordingly, in some embodiments, a subject is identified as having an FGF19 associated cancer if FGF19 is over-expressed in a sample (e.g., a cancer tissue sample or other clinical sample). It should be appreciated that FGF19 expression levels may be difficult to measure accurately in some tissues. Accordingly, a highly-sensitive assay (e.g., a PCR based assay, such as an RT-PCR assay, for example using TaqMan) may be used in some embodiments. In some embodiments, a subject having a high copy number of an 11q13 locus (e.g., 11q13.3, a Cyclin D1 genomic sequence, an FGF19 genomic sequence, or any combination thereof) may be identified as having a cancer associated with FGF19 activity if FGF19 is over-expressed in a tissue sample (e.g., a cancer tissue sample having a high copy number of the 11q13 locus). It should be appreciated that Cyclin D1 or other genomic sequence that is adjacent to the FGF19 gene may be used as a marker of FGF19 amplification if the two genetic loci are sufficiently close to be co-amplified in most examples (e.g., at least 80%, at least 90%, at least 95%, at least 99%, or about 100%) of cancer containing the amplification of the particular 11q13 region. It should be appreciated that a high copy number of an 11q13 locus (e.g., 11q13.3, FGF19, Cyclin D1, etc., or any combination thereof) may be any copy number that is higher than normal (e.g., one or more copies than normal, e.g., a 2 fold increase, a 3 fold increase, a 4 fold increase, or a higher fold increase in copy number relative to the normal number of copies of the genetic locus).

In some embodiments, certain cancers such as liver, esophageal, stomach, and related cancers, may be identified as associated with FGF19 activity if they contain a high copy number of FGF19 genomic sequences, because these cancers are shown herein to be associated with FGF19 over-expression when the FGF19 gene is amplified. In contrast, certain other cancers (e.g., breast, melanoma, lung) are shown herein to contain a high copy number of the FGF19 gene without resulting in FGF19 over-expression. Accordingly, in some embodiments, FGF19 is not a driver of cancer in these tissues. However, in some embodiments, if FGF19 over-expression is detected in certain cancers from these tissues, then FGF19 may be a driver in those cancers.

In some embodiments, a tissue sample may be obtained and assayed to detect FGF19 amplification and/or over-expression as described herein. However, in some embodiments, a patient status may be determined by obtaining (e.g., receiving from a testing laboratory or clinic) information about the patient FGF19 status.

It should be appreciated that when FGF19 is identified as a driver of cancer in a subject, then the subject and/or the cancer are candidates for treatment with an FGF19 inhibitor, for example, an inhibitor of FGF19 expression, activity, or any combination thereof (e.g., an RNA, DNA, antibody, small molecule, or other inhibitor, or any combination thereof, for example as described herein). It should be appreciated that one or more FGF19 inhibitors may be used in combination therapy with one or more other cancer treatment regimes (e.g., chemotherapy, antibody therapy, surgery, radiation, or any combination thereof).

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, cell and cancer biology, virology, immunology, microbiology, genetics and protein and nucleic acid chemistry described herein are those well known and commonly used in the art.

The methods and techniques of the present invention are generally performed according to conventional methods well known in the art and as described in various general and more specific references that are cited and discussed throughout the present specification, unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2003); Harlow and Lane, Antibodies: A Laboratory Manual, Cold Spring Harbor Laboratory Press, Cold Spring Harbor, N.Y. (1990); Coffin et al., Retroviruses, Cold Spring Harbor Laboratory Press; Cold Spring Harbor, N.Y. (1997); Bast et al., Cancer Medicine, 5th ed., Frei, Emil, editors, BC Decker Inc., Hamilton, Canada (2000); Lodish et al., Molecular Cell Biology, 4th ed., W. H. Freeman & Co., New York (2000); Griffiths et al., Introduction to Genetic Analysis, 7th ed., W. H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000). All of the above and any other publications, patents and published patent applications referred to in this application are specifically incorporated by reference herein.

### 1. Diagnostic Methods

FGF19 is part of the 11q13 amplicon, which is one of the most frequently amplified amplicons in many types of cancers—up to 50% of oral carcinomas and 25% of esophageal tumors (Jiang, W., S. M. Kahn, et al. (1992). “Amplification and expression of the human cyclin D gene in esophageal cancer.” Cancer Res 52(10): 2980-3; Huang, X., T. E. Godfrey, et al. (2006). “Comprehensive genome and transcriptome analysis of the 11q13 amplicon in human oral cancer and synteny to the 7F5 amplicon in murine oral carcinoma.” Genes Chromosomes Cancer 45(11): 1058-69), 15% in both breast and liver cancer and from 5% to 30% in lung cancer (Zhang, Y. J., W. Jiang, et al. (1993). “Amplification and over-expression of cyclin D1 in human hepatocellular carcinoma.” Biochem Biophys Res Commun 196(2): 1010-6; Ormandy, C. J., E. A. Musgrove, et al. (2003). “Cyclin D1, EMS1 and 11q13 amplification in breast cancer.” Breast Cancer Res Treat 78(3): 323-35; Gautschi, O., D. Ratschiller, et al. (2007). “Cyclin D1 in non-small cell lung cancer: a key driver of malignant transformation.” Lung Cancer 55(1): 1-14). Previous analysis of expression and oncogenic function by several laboratories has established that CCND1 is a driver gene of the 11q13 amplicon (Ormandy, C. J., E. A. Musgrove, et al. (2003). “Cyclin D1, EMS1 and 11q13 amplification in breast cancer.” Breast Cancer Res Treat 78(3): 323-35). The present invention describes the surprising discovery that the FGF19 gene could be a driver gene for the 11q13 amplicon in liver and esophageal cancer, but not lung, melanoma or breast cancer. Accordingly the invention provides diagnostic methods of detecting human cancer or susceptibility to cancer based on copy number amplification of a nucleotide sequence comprising the FGF19 gene (e.g., the 11q13 locus) and based on expression of the FGF19 gene (protein, mRNA).

The amplified genomic region is also called an amplicon. In some embodiments, since the FGF19 gene is the driver oncogene of the 11q13 amplicon, preferred markers for the diagnostic methods of the present invention are nucleotide sequences from the FGF19 gene (e.g., for detecting increased copy number or for detecting increased mRNA expression relative to normal copy number or expression levels). In some embodiments, nucleotide sequences adjacent to or surrounding the FGF19 gene may be used as diagnostic markers (e.g., to detect increased copy number of a genomic locus containing the FGF19 gene). In some embodiments, the FGF19 protein or one or more FGF19 specific peptides may be detected (e.g., using an antibody or antigen-binding fragment thereof) as markers of FGF19 expression levels (e.g., to detect FGF19 over-expression). An exemplary human FGF19 coding sequence has the following nucleotide sequence:

(SEQ ID NO: 1; GenBank No. NM_005117.2)
gctcccagcc aagaacctcg gggccgctgc gcggtgggga ggagttcccc gaaacccggc
cgctaagcga ggcctcctcc tcccgcagat ccgaacggcc tgggcggggt caccccggct
gggacaagaa gccgccgcct gcctgcccgg gcccggggag ggggctgggg ctggggccgg
aggcggggtg tgagtgggtg tgtgcggggg gcggaggctt gatgcaatcc cgataagaaa
tgctcgggtg tcttgggcac ctacccgtgg ggcccgtaag gcgctactat ataaggctgc
cggcccggag ccgccgcgcc gtcagagcag gagcgctgcg tccaggatct agggccacga
ccatcccaac ccggcactca cagccccgca gcgcatcccg gtcgccgccc agcctcccgc
acccccatcg ccggagctgc gccgagagcc ccagggaggt gccatgcgga gcgggtgtgt
ggtggtccac gtatggatcc tggccggcct ctggctggcc gtggccgggc gccccctcgc
cttctcggac gcggggcccc acgtgcacta cggctggggc gaccccatcc gcctgcggca
cctgtacacc tccggccccc acgggctctc cagctgcttc ctgcgcatcc gtgccgacgg
cgtcgtggac tgcgcgcggg gccagagcgc gcacagtttg ctggagatca aggcagtcgc
tctgcggacc gtggccatca agggcgtgca cagcgtgcgg tacctctgca tgggcgccga
cggcaagatg caggggctgc ttcagtactc ggaggaagac tgtgctttcg aggaggagat
ccgcccagat ggctacaatg tgtaccgatc cgagaagcac cgcctcccgg tctccctgag
cagtgccaaa cagcggcagc tgtacaagaa cagaggcttt cttccactct ctcatttcct
gcccatgctg cccatggtcc cagaggagcc tgaggacctc aggggccact tggaatctga
catgttctct tcgcccctgg agaccgacag catggaccca tttgggcttg tcaccggact
ggaggccgtg aggagtccca gctttgagaa gtaactgaga ccatgcccgg gcctcttcac
tgctgccagg ggctgtggta cctgcagcgt gggggacgtg cttctacaag aacagtcctg
agtccacgtt ctgtttagct ttaggaagaa acatctagaa gttgtacata ttcagagttt
tccattggca gtgccagttt ctagccaata gacttgtctg atcataacat tgtaagcctg
tagcttgccc agctgctgcc tgggccccca ttctgctccc tcgaggttgc tggacaagct
gctgcactgt ctcagttctg cttgaatacc tccatcgatg gggaactcac ttcctttgga
aaaattctta tgtcaagctg aaattctcta attttttctc atcacttccc caggagcagc
cagaagacag gcagtagttt taatttcagg aacaggtgat ccactctgta aaacagcagg
taaatttcac tcaaccccat gtgggaattg atctatatct ctacttccag ggaccatttg
cccttcccaa atccctccag gccagaactg actggagcag gcatggccca ccaggcttca
ggagtagggg aagcctggag ccccactcca gccctgggac aacttgagaa ttccccctga
ggccagttct gtcatggatg ctgtcctgag aataacttgc tgtcccggtg tcacctgctt
ccatctccca gcccaccagc cctctgccca cctcacatgc ctccccatgg attggggcct
cccaggcccc ccaccttatg tcaacctgca cttcttgttc aaaaatcagg aaaagaaaag
atttgaagac cccaagtctt gtcaataact tgctgtgtgg aagcagcggg ggaagaccta
gaaccctttc cccagcactt ggttttccaa catgatattt atgagtaatt tattttgata
tgtacatctc ttattttctt acattattta tgcccccaaa ttatatttat gtatgtaagt
gaggtttgtt ttgtatatta aaatggagtt tgtttgtaaa aaaaaaaaaa aaaaaaa

An exemplary FGF19 protein has the following polypeptide sequence:

(SEQ ID NO: 2; GenBank No. NP_005108.1)
mrsgcvvvhv wilaglwlav agrplafsda gphvhygwgd pirlrhlyts gphglsscfl
riradgvvdc argqsahsll eikavalrtv aikgvhsvry lcmgadgkmq gllqyseedc
afeeeirpdg ynvyrsekhr lpvslssakq rqlyknrgfl plshflpmlp mvpeepedlr
ghlesdmfss pletdsmdpf glvtgleavr spsfek

Other sequences comprising the human FGF19 nucleotide sequence include GenBank Nos. CH471076.1, NW_{—}925106.1, NT_{—}078088.3, BC017664.1, AY891563.1, AY888961.1, AY888960.1, AY358302.1, AF110400.1. Other human polypeptide sequences include GenBank Nos. AAQ88669.1, 095750.1, EAW74751.1, AAH17664.1, AAD45973.1. FGF19 orthologs in other animal species have also been identified; their sequences include GenBank Nos. NM_{—}001012246.1 and NP_{—}001012246.1 (zebrafish), NM_{—}204674.1 and NP_{—}990005.1 (chicken), and XP_{—}001100825 (rhesus monkey).

Detection of FGF19 amplification (e.g., 11q13 amplification) is also useful in stratifying cancer patients for their responsiveness to FGF19-based treatment. In some cases, detection of FGF19 amplification in combination with detection of FGF19 over-expression is useful in stratifying cancer patients. It is well known in cancer biology that not all patients of a particular type of cancer respond in the same way to a given therapy, and that responsiveness to a therapy is related at least in part to the genetic makeup of the cancer. A pre-treatment assessment of a patient of cancer genetic characteristics will therefore improve the success rate of the treatment. Given that an amplification of the FGF19 gene exists in only a subset of cancer patients, it is useful to confirm its existence in patients who are to receive a cancer therapy based on an FGF19 inhibitor.

In certain embodiments, the invention provides a method for selecting a patient for treatment with an inhibitor of FGF19. In other embodiments, the invention provides a method for diagnosing a patient having or at risk for developing cancer, such as liver or esophageal cancer. Such methods comprise detecting, in a DNA sample from a patient, amplification of a FGF19 gene, or a portion thereof. Amplification of the FGF19 gene indicates that the patient is a candidate for treatment with an inhibitor of FGF19, or indicates that the patient has or is at risk for developing cancer.

Any assay that permits detection of amplification of the FGF19 gene may be used. Such methods may encompass, for example, DNA sequencing, hybridization, ligation, PCR, or primer extension methods. Furthermore, any combination of these methods may be utilized in the invention.

In one embodiment, amplification of the FGF19 gene is detected and/or determined by hybridization. In one embodiment, a polynucleotide primer or probe hybridizes to the FGF19 gene and/or flanking nucleotides. For example, the primer or probe can hybridize to a genomic sequence that encodes the entirety or part (e.g., at 15, 20, 25, 30, 35, 40, 45, 50, or more than 50 contiguous nucleotides) of the FGF19 gene, including allelic variants of the gene. The polynucleotide probe may comprise nucleotides that are fluorescently, radioactively, or chemically labeled to facilitate detection of hybridization. Hybridization may be performed and detected by standard methods known in the art, such as by Northern blotting, Southern blotting, fluorescent in situ hybridization (FISH), or by hybridization to polynucleotides immobilized on a solid support, such as a DNA array or microarray. In certain embodiments, comparative genomic hybridization is used to detect amplification of the FGF19 gene. Preferably, the primers and probes hybridize to a relatively unique part of the gene so as to reduce background noise signal. Genomic DNA from a normal individual or from a healthy tissue of the cancer patient can be used as a control for detecting amplification.

It is well known in the art how to perform hybridization experiments with nucleic acid molecules. The skilled artisan is familiar with the hybridization conditions required in the present invention and understands readily that appropriate stringency conditions which promote DNA hybridization can be varied. Such hybridization conditions are referred to in standard text books, such as Molecular Cloning: A Laboratory Manual, Cold Spring Harbor Laboratory (2001); and Current Protocols in Molecular Biology, eds. Ausubel et al., John Wiley & Sons (1992). Particularly useful in methods of the present invention are polynucleotides which are capable of hybridizing to a FGF19 gene or portion, under stringent conditions. Under stringent conditions, a polynucleotide that hybridizes to a FGF19 gene detectably and specifically binds to the FGF19 gene. Polynucleotides, oligonucleotides and fragments thereof in accordance with the invention selectively hybridize to nucleic acid strands under hybridization and wash conditions that minimize appreciable amounts of detectable binding to nonspecific nucleic acids.

Nucleic acid hybridization is affected by such conditions as salt concentration, temperature, organic solvents, base composition, length of the complementary strands, and the number of nucleotide base mismatches between the hybridizing nucleic acids, as will readily be appreciated by those skilled in the art. Stringent temperature conditions will generally include temperatures in excess of 30° C., or may be in excess of 37° C. or 45° C. Stringency increases with temperature. For example, temperatures greater than 45° C. are highly stringent conditions. Stringent salt conditions will ordinarily be less than 1000 mM, or may be less than 500 mM or 200 mM. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. Particularly useful in methods of the present invention are polynucleotides which are capable of hybridizing to a FGF19 gene or portion, under stringent conditions. It is understood, however, that the appropriate stringency conditions may be varied in the present invention to promote DNA hybridization. In certain embodiments, polynucleotides of the present invention hybridize to a FGF19 gene or portion, under highly stringent conditions. In one embodiment, the invention provides nucleic acids which hybridize under low stringency conditions of 6.0×SSC at room temperature followed by a wash at 2.0×SSC at room temperature. The combination of parameters, however, is much more important than the measure of any single parameter. See, e.g., Wetmur and Davidson, 1968. Probe sequences may also hybridize specifically to duplex DNA under certain conditions to form triplex or higher order DNA complexes. The preparation of such probes and suitable hybridization conditions are well known in the art. One method for obtaining DNA encoding the biosynthetic constructs disclosed herein is by assembly of synthetic oligonucleotides produced in a conventional, automated, oligonucleotide synthesizer.

As used herein, the terms “DNA array,” and “microarray” refer to an ordered arrangement of hybridizable array elements. The array elements are arranged so that there are preferably at least one or more different array elements immobilized on a substrate surface. The hybridization signal from each of the array elements is individually distinguishable. In a preferred embodiment, the array elements comprise polynucleotides, although the present invention could also be used with cDNA or other types of nucleic acid array elements. In one embodiment, genomic profiling as done by microarray analysis is used to detect DNA amplifications. One such method is Representational Oligonucleotide Microarray Analysis (ROMA), a genome-wide scanning method capable of identifying copy number alterations in cells at high resolution (Lucito et al., 2003, Genome Res. 13:2291-2305; Sebat et al., 2004, Science 305:525-528). One can also use, e.g., the GENECHIP arrays, the SNP arrays, and the EXON arrays available from Affymetrix to perform microarray analysis of genomic profiles for detecting FGF19 amplification.

In a specific embodiment, amplification of the FGF19 gene is detecting by hybridizing a polynucleotide probe to genomic DNA by FISH. FISH can be used, for example, in metaphase cells, to detect a deletion or amplification in genomic DNA. Genomic DNA is denatured to separate the complimentary strands within the DNA double helix structure. The polynucleotide probe of the invention is then added to the denatured genomic DNA. The probe signal (e.g., fluorescence) can then be detected (e.g., with a fluorescent microscope) for the level of signal from the hybridized probe. In another specific embodiment, a labeled polynucleotide probe is applied to immobilized polynucleotides on a DNA array. Hybridization may be detected, for example, by measuring the intensity of the labeled probe remaining on the DNA array after washing.

In another embodiment, amplification of the FGF19 gene is detected and/or determined by ligation. In one embodiment, a polynucleotide primer hybridizes to a to one side of the FGF19 gene, or to a portion of the FGF19 gene. A second polynucleotide that hybridizes to a region of the FGF19 gene immediately adjacent to the first primer is also provided. One, or both, of the polynucleotide primers may be fluorescently, radioactively, or chemically labeled. Ligation of the two polynucleotide primers will occur in the presence of DNA ligase if the FGF19 gene is amplified in the sample. Ligation may be detected by gel electrophoresis, mass spectrometry, or by measuring the intensity of fluorescent, radioactive, or chemical labels.

In another embodiment, amplification of the FGF19 gene is detected and/or determined by multiplex ligation-dependent probe amplification (MLPA). MLPA is a technique used to detect chromosomal abnormalities, such as gene amplifications (Schouten et al., 2002, Nucleic Acids Res. 30:e57). In one embodiment, a polynucleotide probe hybridizes to a genomic sequence that encodes the entirety or part (e.g., at 15, 20, 25, 30, 35, 40, 45, 50, 75, 100, 200, 300, 500, 750, 1000, or more than 1000 contiguous nucleotides) of the FGF19 gene, including allelic variants of the gene. A second polynucleotide probe hybridizes adjacent to the first probe. In a specific embodiment, the two probes hybridize to a genomic sequence encoding the FGF19 gene, or a portion of the FGF19 gene. The two adjacent probes can be ligated to each other by ligase to form a target probe. In certain embodiments, the target probe is amplified by PCR amplification using polynucleotide primers specific to the target probe. The amount of target probe is then quantified and correlated with the level of FGF19 gene amplification in the sample. Standard methods for quantifying nucleic acids may be used to quantify the amount of the target probe. For example, electrophoresis may be used to separate a target probe from other DNA in the sample. The target probe can be fluorescently, radioactively, or chemically labeled.

In one embodiment, amplification of the FGF19 gene is detected and/or determined by DNA sequencing. DNA sequence determination may be performed by standard methods such as dideoxy chain termination technology and gel-electrophoresis, or by other methods such as by pyrosequencing (Biotage AB, Uppsala, Sweden). For example, DNA sequencing by dideoxy chain termination may be performed using unlabeled primers and labeled (e.g., fluorescent or radioactive) terminators. Alternatively, sequencing may be performed using labeled primers and unlabeled terminators. The nucleic acid sequence of the DNA in the sample can be compared to the nucleic acid sequence of wildtype DNA to identify whether the FGF19 gene is amplified.

In another embodiment, amplification of the FGF19 gene is detected and/or determined by primer extension with DNA polymerase. In one embodiment, a polynucleotide primer hybridizes to one side of the FGF19 gene, or to a portion of the FGF19 gene. The primer undergoes a primer extension reaction with DNA polymerase. The primers and/or nucleotides may further include fluorescent, radioactive, or chemical probes. A primer labeled by primer extension may be detected by measuring the intensity of the extension product, such as by gel electrophoresis, mass spectrometry, or any other method for detecting fluorescent, radioactive, or chemical labels.

Methods of detecting amplification of the FGF19 gene may include amplification of a region of DNA that comprises the amplified FGF19 gene. Any method of amplification may be used. In one specific embodiment, a region of DNA comprising the FGF19 gene is amplified by using polymerase chain reaction (PCR). PCR was initially described by Mullis (See e.g., U.S. Pat. Nos. 4,683,195 4,683,202, and 4,965,188, herein incorporated by reference), which describes a method for increasing the concentration of a region of DNA, in a mixture of genomic DNA, without cloning or purification. Other PCR methods may also be used for nucleic acid amplification, including but not limited to RT-PCR, quantitative PCR, real time PCR, Rapid Amplified Polymorphic DNA Analysis, Rapid Amplification of cDNA Ends (RACE), or rolling circle amplification. RT-PCR is described, for example, in Nistor et al., 2006, BMC Clinical Pathology, 6:2). For example, polynucleotide primers are combined with a DNA sample taken from a patient (or any polynucleotide sequence that can be amplified with polynucleotide primers), wherein the DNA comprises the FGF19 gene. The mixture also includes the necessary amplification reagents (e.g., deoxyribonucleotide triphosphates, buffer, etc.) necessary for the thermal cycling reaction. According to standard PCR methods, the mixture undergoes a series of denaturation, primer annealing, and polymerase extension steps to amplify the region of DNA that comprises the FGF19 gene. The length of the amplified region of DNA is determined by the relative positions of the primers with respect to each other, and therefore, this length is a controllable parameter. For example, hybridization of the primers may occur such that the ends of the primers proximal to the variation are separated by 1 to 10,000 base pairs (e.g., 10 base pairs (bp) 50 bp, 200 bp, 500 bp, 1,000 bp, 2,500 bp, 5,000 bp, or 10,000 bp).

Standard instrumentation known to those skilled in the art are used for the amplification and detection of amplified DNA. For example, a wide variety of instrumentation has been developed for carrying out nucleic acid amplifications, particularly PCR, e.g. Johnson et al, U.S. Pat. No. 5,038,852 (computer-controlled thermal cycler); Wittwer et al, Nucleic Acids Research, 17: 4353-4357 (1989) (capillary tube PCR); Hallsby, U.S. Pat. No. 5,187,084 (air-based temperature control); Garner et al, Biotechniques, 14: 112-115 (1993)(high-throughput PCR in 864-well plates); Wilding et al, International application No. PCT/US93/04039 (PCR in micro-machined structures); Schnipelsky et al, European patent application No. 90301061.9 (publ. No. 0381501 A2)(disposable, single use PCR device), and the like.

In one embodiment, the amplified DNA is analyzed in conjunction with one of the detection methods described herein, such as by DNA sequencing. The amplified DNA may alternatively be analyzed by hybridization with a labeled probe, hybridization to a DNA array or microarray, by incorporation of biotinylated primers followed by avidin-enzyme conjugate detection, or by incorporation of ³²P-labeled deoxynucleotide triphosphates, such as dCTP or dATP, into the amplified segment. In a specific embodiment, the amplified DNA is analyzed by determining the length of the amplified DNA by electrophoresis or chromatography. For example, the amplified DNA is analyzed by gel electrophoresis. Methods of gel electrophoresis are well known in the art. See for example, Current Protocols in Molecular Biology, eds. Ausubel et al., John Wiley & Sons: 1992. The amplified DNA can be visualized, for example, by fluorescent or radioactive means, or with other dyes or markers that intercalate DNA. The DNA may also be transferred to a solid support such as a nitrocellulose membrane and subjected to Southern Blotting following gel electrophoresis. In one embodiment, the DNA is exposed to ethidium bromide and visualized under ultra-violet light.

In order to determine FGF19 gene expression, and detect over-expression, protein or mRNA levels may be measured.

FGF19 protein levels may be measured by any appropriate method known in the art, such as western analysis. Other methods known to one of ordinary skill in the art may be employed to analyze proteins levels, for example immunohistochemistry, immunocytochemistry, ELISA, Radioimmunoassays, proteomics methods, such as mass spectroscopy or antibody arrays. Other appropriate methods will be apparent to the skilled artisan.

In some cases, it may be beneficial to use a commercially available antibody raised against FGF19 to measure FGF19 protein levels. Such antibodies include, for example, Goat Anti-Human FGF19 Polyclonal Antibody, Unconjugated from Abcam; Mouse Anti-Human FGF19 MaxPab® Polyclonal Antibody, Unconjugated from Abnova Corporation; Mouse Anti-Human FGF19 MaxPab® Polyclonal Antibody, Unconjugated from Abnova Corporation; Goat anti-Human FGF19 Polyclonal Antibody, Unconjugated from LifeSpan BioSciences; Goat Anti-Human FGF19 Polyclonal Antibody, Unconjugated from MBL International; Mouse Anti-Human FGF19 Maxpab Polyclonal Antibody, Unconjugated from Novus Biologicals; Mouse Anti-Human FGF19 Monoclonal Antibody, Unconjugated, Clone 4C4 from Novus Biologicals; Mouse Anti-Human FGF19 Polyclonal Antibody, Unconjugated from Novus Biologicals. Other antibodies are known in the art (See, for example, US Patent Application 20070248604 and the anti-FGF19 antibodies disclosed therein, e.g., anti-FGF19 mab 1D1 and anti-FGF19 mab 1A6. The contents of this application are incorporated herein by reference in their entirety)

FGF19 mRNA levels may be measured by any appropriate method known in the art, for example, RT-PCR, northern analysis, RNAse protection assay, microarray analysis, cDNA array analysis, bead array analysis, FISH analysis. Other appropriate methods will be apparent to the skilled artisan.

### 2. Therapeutic Modalities

The present invention provides methods of targeted therapies for a variety of cancers, including lung and esophageal cancers. Targeted therapies are based on the premise that cancer cells require continuous oncogenic signaling for survival and proliferation. Thus, drugs that terminate or disrupt this signaling remove the stimuli for cancer growth. A targeted therapy directly interferes with a driver oncogene and can be effected by, e.g., monoclonal antibodies (humanized or chimeric), cancer vaccines, and gene therapy (including RNA interference, antisense and ribozyme technology).

The present discovery that FGF19 is a driver oncogene in the 11q13 amplicon provides a rationale for targeting FGF19 in cancer therapy, at least for patients that have an amplification of the FGF19 gene. Inhibitors of FGF19 are discussed below.

Therapies that may be tested and evaluated in the methods and models of this invention include both general and targeted therapies. As used herein, a general therapy can be, for example, a pharmaceutical or chemical with physiological effects, such as pharmaceuticals that have been used in chemotherapy for cancer. Chemotherapeutic agents inhibit proliferation of tumor cells, and generally interfere with DNA replication or cellular metabolism. See, e.g., The McGraw-Hill Dictionary of Chemical Terms (Parker, S., Ed., McGraw-Hill, San Francisco (1985)). Chemotherapeutic agents may or may not have been characterized for their target of action in cells. However, this invention and its methods and models allow evaluation of such therapies for defined genetic alterations.

FGF19 targeted therapy can be used in conjunction with a traditional cancer therapy that targets growth factors and their receptors such as epidermal growth factor receptor (EGFR) (e.g., a Gefitinib, Erlotinib, or Imatinib therapy) or vascular epidermal growth factor (VEGF) (e.g., bevacizumab (AVASTIN®)). The targeted therapy of this invention can also be used in conjunction with chemotherapies such as Taxanes (a group of drugs that includes paclitaxel (TAXOL®) and docetaxel (TAXOTERE®)), Cisplatinin, Methotrexate, and 5-fluorouracil. The targeted therapy and combination therapy of this invention will inhibit growth of cancer cells and can reduce tumor size, cause tumor regression, prevention of metastasis, and prevention of angiogenesis at tumor sites.

The targeted therapy of this invention is particularly useful in treating liver and esophageal cancers, where FGF19 amplification has been observed to occur at a relatively high rate. Cancers treatable by the therapy include malignant tumors, pre-malignant conditions such as proliferative and cellular hyperplasia, neoplasm, and metastasized cancer.

Antisense Polynucleotides

In certain embodiments, the invention provides polynucleotides that comprise an antisense sequence that acts through an antisense mechanism for inhibiting expression of the FGF19 gene. Antisense technologies have been widely utilized to regulate gene expression (Buskirk et al., Chem Biol 11, 1157-63 (2004); and Weiss et al., Cell Mol Life Sci 55, 334-58 (1999)). As used herein, “antisense” technology refers to administration or in situ generation of molecules or their derivatives which specifically hybridize (e.g., bind) under cellular conditions, with the target nucleic acid of interest (mRNA and/or genomic DNA) encoding one or more of the target proteins so as to inhibit expression of that protein, e.g., by inhibiting transcription and/or translation, such as by steric hindrance, altering splicing, or inducing cleavage or other enzymatic inactivation of the transcript. The binding may be by conventional base pair complementarity, or, for example, in the case of binding to DNA duplexes, through specific interactions in the major groove of the double helix. In general, “antisense” technology refers to the range of techniques generally employed in the art, and includes any therapy that relies on specific binding to nucleic acid sequences.

A polynucleotide that comprises an antisense sequence of the present invention can be delivered, for example, as a component of an expression plasmid which, when transcribed in the cell, produces a nucleic acid sequence that is complementary to at least a unique portion of the target nucleic acid. Alternatively, the polynucleotide that comprises an antisense sequence can be generated outside of the target cell, and which, when introduced into the target cell causes inhibition of expression by hybridizing with the target nucleic acid. Polynucleotides of the invention may be modified so that they are resistant to endogenous nucleases, e.g. exonucleases and/or endonucleases, and are therefore stable in vivo. Examples of nucleic acid molecules for use in polynucleotides of the invention are phosphoramidate, phosphorothioate and methylphosphonate analogs of DNA (see also U.S. Pat. Nos. 5,176,996; 5,264,564; and 5,256,775). General approaches to constructing polynucleotides useful in antisense technology have been reviewed, for example, by van der krol et al. (1988) Biotechniques 6:958-976; and Stein et al. (1988) Cancer Res 48:2659-2668.

Antisense approaches involve the design of polynucleotides (either DNA or RNA) that are complementary to a target nucleic acid encoding a FGF19 gene. The antisense polynucleotide may bind to an mRNA transcript and prevent translation of a protein of interest. Absolute complementarity, although preferred, is not required. In the case of double-stranded antisense polynucleotides, a single strand of the duplex DNA may thus be tested, or triplex formation may be assayed. The ability to hybridize will depend on both the degree of complementarity and the length of the antisense sequence. Generally, the longer the hybridizing nucleic acid, the more base mismatches with a target nucleic acid it may contain and still form a stable duplex (or triplex, as the case may be). One skilled in the art can ascertain a tolerable degree of mismatch by use of standard procedures to determine the melting point of the hybridized complex.

Antisense polynucleotides that are complementary to the 5′ end of an mRNA target, e.g., the 5′ untranslated sequence up to and including the AUG initiation codon, should work most efficiently at inhibiting translation of the mRNA. However, sequences complementary to the 3′ untranslated sequences of mRNAs have recently been shown to be effective at inhibiting translation of mRNAs as well (Wagner, R. 1994. Nature 372:333). Therefore, antisense polynucleotides complementary to either the 5′ or 3′ untranslated, non-coding regions of a FGF19 gene could be used in an antisense approach to inhibit translation of a FGF19 gene. Antisense polynucleotides complementary to the 5′ untranslated region of an mRNA should include the complement of the AUG start codon. Antisense polynucleotides complementary to mRNA coding regions are less efficient inhibitors of translation but could also be used in accordance with the invention. Whether designed to hybridize to the 5′, 3′, or coding region of mRNA, antisense polynucleotides should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense polynucleotide to inhibit expression of a FGF19 gene. It is preferred that these studies utilize controls that distinguish between antisense gene inhibition and nonspecific biological effects of antisense polynucleotide. It is also preferred that these studies compare levels of the target RNA or protein with that of an internal control RNA or protein. Additionally, it is envisioned that results obtained using the antisense polynucleotide are compared with those obtained using a control antisense polynucleotide. It is preferred that the control antisense polynucleotide is of approximately the same length as the test antisense polynucleotide and that the nucleotide sequence of the control antisense polynucleotide differs from the antisense sequence of interest no more than is necessary to prevent specific hybridization to the target sequence.

Polynucleotides of the invention, including antisense polynucleotides, can be DNA or RNA or chimeric mixtures or derivatives or modified versions thereof, single-stranded or double-stranded. Polynucleotides of the invention can be modified at the base moiety, sugar moiety, or phosphate backbone, for example, to improve stability of the molecule, hybridization, etc. Polynucleotides of the invention may include other appended groups such as peptides (e.g., for targeting host cell receptors), or agents facilitating transport across the cell membrane (see, e.g., Letsinger et al., 1989, Proc Natl Acad. Sci. USA 86:6553-6556; Lemaitre et al., 1987, Proc Natl Acad. Sci. USA 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, Pharm. Res. 5:539-549 (1988)). To this end, a polynucleotide of the invention may be conjugated to another molecule, e.g., a peptide, hybridization triggered cross-linking agent, transport agent, hybridization-triggered cleavage agent, etc.

Polynucleotides of the invention, including antisense polynucleotides, may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl)uracil, (acp3)w, and 2,6-diaminopurine.

Polynucleotides of the invention may also comprise at least one modified sugar moiety selected from the group including but not limited to arabinose, 2-fluoroarabinose, xylulose, and hexose.

A polynucleotide of the invention can also contain a neutral peptide-like backbone. Such molecules are termed peptide nucleic acid (PNA)-oligomers and are described, e.g., in Perry-O'Keefe et al. (1996) Proc. Natl. Acad. Sci. USA 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, a polynucleotide of the invention comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, polynucleotides of the invention, including antisense polynucleotides are -anomeric oligonucleotides. An -anomeric oligonucleotide forms specific double-stranded hybrids with complementary RNA in which, contrary to the usual-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Polynucleotides of the invention, including antisense polynucleotides, may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. Nucl. Acids Res. 16:3209 (1988)), methylphosphonate oligonucleotides can be prepared by use of controlled pore glass polymer supports (Sarin et al., Proc. Natl. Acad. Sci. USA 85:7448-7451 (1988)), etc.

While antisense sequences complementary to the coding region of an mRNA sequence can be used, those complementary to the transcribed untranslated region and to the region comprising the initiating methionine are most preferred.

Antisense polynucleotides can be delivered to cells that express target genes in vivo. A number of methods have been developed for delivering nucleic acids into cells; e.g., they can be injected directly into the tissue site, or modified nucleic acids, designed to target the desired cells (e.g., antisense polynucleotides linked to peptides or antibodies that specifically bind receptors or antigens expressed on the target cell surface) can be administered systematically.

However, it may be difficult to achieve intracellular concentrations of the antisense polynucleotides sufficient to attenuate the activity of a FGF19 gene or mRNA in certain instances. Therefore, another approach utilizes a recombinant DNA construct in which the antisense polynucleotide is placed under the control of a strong pol III or pol II promoter. The use of such a construct to transfect target cells in the patient will result in the transcription of sufficient amounts of antisense polynucleotides that will form complementary base pairs with the FGF19 gene or mRNA and thereby attenuate the activity of FGF19 protein. For example, a vector can be introduced in vivo such that it is taken up by a cell and directs the transcription of an antisense polynucleotide that targets a FGF19 gene or mRNA. Such a vector can remain episomal or become chromosomally integrated, as long as it can be transcribed to produce the desired antisense polynucleotide. Such vectors can be constructed by recombinant DNA technology methods standard in the art. Vectors can be plasmid, viral, or others known in the art, used for replication and expression in mammalian cells. A promoter may be operably linked to the sequence encoding the antisense polynucleotide. Expression of the sequence encoding the antisense polynucleotide can be by any promoter known in the art to act in mammalian, preferably human cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionine gene (Brinster et al, Nature 296:3942 (1982)), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi Constructs—siRNAs and miRNAs

RNA interference (RNAi) is a phenomenon describing double-stranded (ds)RNA-dependent gene specific posttranscriptional silencing. Initial attempts to harness this phenomenon for experimental manipulation of mammalian cells were foiled by a robust and nonspecific antiviral defense mechanism activated in response to long dsRNA molecules. Gil et al. Apoptosis 2000, 5:107-114. The field was significantly advanced upon the demonstration that synthetic duplexes of 21 nucleotide RNAs could mediate gene specific RNAi in mammalian cells, without invoking generic antiviral defense mechanisms. Elbashir et al. Nature 2001, 411:494-498; Caplen et al. Proc Natl Acad Sci 2001, 98:9742-9747. As a result, small-interfering RNAs (siRNAs) and micro RNAs (miRNAs) have become powerful tools to dissect gene function. The chemical synthesis of small RNAs is one avenue that has produced promising results. Numerous groups have also sought the development of DNA-based vectors capable of generating such siRNA within cells. Several groups have recently attained this goal and published similar strategies that, in general, involve transcription of short hairpin (sh)RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes.

Accordingly, the present invention provides a polynucleotide comprising an RNAi sequence that acts through an RNAi or miRNA mechanism to attenuate expression of a FGF19 gene. For instance, a polynucleotide of the invention may comprise a miRNA or siRNA sequence that attenuates or inhibits expression of a FGF19 gene. In one embodiment, the miRNA or siRNA sequence is between about 19 nucleotides and about 75 nucleotides in length, or preferably, between about 25 base pairs and about 35 base pairs in length. In certain embodiments, the polynucleotide is a hairpin loop or stem-loop that may be processed by RNAse enzymes (e.g., Drosha and Dicer).

An RNAi construct contains a nucleotide sequence that hybridizes under physiologic conditions of the cell to the nucleotide sequence of at least a portion of the mRNA transcript for a FGF19 gene. The double-stranded RNA need only be sufficiently similar to natural RNA that it has the ability to mediate RNAi. The number of tolerated nucleotide mismatches between the target sequence and the RNAi construct sequence is no more than 1 in 5 basepairs, or 1 in 10 basepairs, or 1 in 20 basepairs, or 1 in 50 basepairs. It is primarily important the that RNAi construct is able to specifically target a FGF19 gene. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of polynucleotides comprising RNAi sequences can be carried out by any of the methods for producing polynucleotides described herein. For example, polynucleotides comprising RNAi sequences can be produced by chemical synthetic methods or by recombinant nucleic acid techniques. Endogenous RNA polymerase of the treated cell may mediate transcription in vivo, or cloned RNA polymerase can be used for transcription in vitro. Polynucleotides of the invention, including wildtype or antisense polynucleotides, or those that modulate target gene activity by RNAi mechanisms, may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. Polynucleotides of the invention may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “siRNAs.” These nucleic acids are between about 19-35 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex or translation is inhibited. In a particular embodiment, the 21-23 nucleotide siRNA molecules comprise a 3′ hydroxyl group.

In other embodiments, the subject RNAi constructs are “miRNAs.” microRNAs (miRNAs) are small non-coding RNAs that direct post transcriptional regulation of gene expression through interaction with homologous mRNAs. miRNAs control the expression of genes by binding to complementary sites in target mRNAs from protein coding genes. miRNAs are similar to siRNAs. miRNAs are processed by nucleolytic cleavage from larger double-stranded precursor molecules. These precursor molecules are often hairpin structures of about 70 nucleotides in length, with 25 or more nucleotides that are base-paired in the hairpin. The RNAse III-like enzymes Drosha and Dicer (which may also be used in siRNA processing) cleave the miRNA precursor to produce an miRNA. The processed miRNA is single-stranded and incorporates into a protein complex, termed RISC or miRNP. This RNA-protein complex targets a complementary mRNA. miRNAs inhibit translation or direct cleavage of target mRNAs. Brennecke et al., Genome Biology 4:228 (2003); Kim et al., Mol. Cells. 19:1-15 (2005).

In certain embodiments, miRNA and siRNA constructs can be generated by processing of longer double-stranded RNAs, for example, in the presence of the enzymes Dicer or Drosha. Dicer and Drosha are RNAse III-like nucleases that specifically cleave dsRNA. Dicer has a distinctive structure which includes a helicase domain and dual RNAse III motifs. Dicer also contains a region of homology to the RDE1/QDE2/ARGONAUTE family, which have been genetically linked to RNAi in lower eukaryotes. Indeed, activation of, or over-expression of Dicer may be sufficient in many cases to permit RNA interference in otherwise non-receptive cells, such as cultured eukaryotic cells, or mammalian (non-oocytic) cells in culture or in whole organisms. Methods and compositions employing Dicer, as well as other RNAi enzymes, are described in U.S. Pat. App. Publication No. 2004/0086884.

In one embodiment, the Drosophila in vitro system is used. In this embodiment, a polynucleotide comprising an RNAi sequence or an RNAi precursor is combined with a soluble extract derived from Drosophila embryo, thereby producing a combination. The combination is maintained under conditions in which the dsRNA is processed to RNA molecules of about 21 to about 23 nucleotides.

The miRNA and siRNA molecules can be purified using a number of techniques known to those of skill in the art. For example, gel electrophoresis can be used to purify such molecules. Alternatively, non-denaturing methods, such as non-denaturing column chromatography, can be used to purify the siRNA and miRNA molecules. In addition, chromatography (e.g., size exclusion chromatography), glycerol gradient centrifugation, affinity purification with antibody can be used to purify siRNAs and miRNAs.

In certain embodiments, at least one strand of the siRNA sequence of an effector domain has a 3′ overhang from about 1 to about 6 nucleotides in length, or from 2 to 4 nucleotides in length. In other embodiments, the 3′ overhangs are 1-3 nucleotides in length. In certain embodiments, one strand has a 3′ overhang and the other strand is either blunt-ended or also has an overhang. The length of the overhangs may be the same or different for each strand. In order to further enhance the stability of the siRNA sequence, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In certain embodiments, a polynucleotide of the invention that comprises an RNAi sequence or an RNAi precursor is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that miRNAs and siRNAs can be produced by processing a hairpin RNA in the cell.

In yet other embodiments, a plasmid is used to deliver the double-stranded RNA, e.g., as a transcriptional product. After the coding sequence is transcribed, the complementary RNA transcripts base-pair to form the double-stranded RNA.

In some embodiments, the invention relates to the following siRNA and shRNA sequences. It should be appreciated that these sequences and related sequences may be used in any appropriate methods of the invention (e.g., in therapeutic methods described herein).

| Cyclin D1 (CCND1) Targeting Oligonucleotides Based on NM_053056 | |
|---|---|
| CND1K1: (19mer Sense Strand) GGGTATGTTTAATCTGTTA | (SEQ ID NO: 3) |
| CND1K1: (22mer Sense Strand) TTGGGTATGTTTAATCTGTTAT | (SEQ ID NO: 4) |
| CND1K1: (shRNA) | |
| TGCTGTTGACAGTGAGCGCTGGGTATGTTTAATCTGTTATTAGTGAAGCCA | (SEQ ID NO: 5) |
| CAGATGTAATAACAGATTAAACATACCCAATGCCTACTGCCTCGGA | |
| CND1K2: (19mer Sense Strand) GCATGTAGTCACTTTATAA | (SEQ ID NO: 6) |
| CND1K2: (22mer Sense Strand) ATGCATGTAGTCACTTTATAAG | (SEQ ID NO: 7) |
| CND1K2: (shRNA) | |
| TGCTGTTGACAGTGAGCGCTGCATGTAGTCACTTTATAAGTAGTGAAGCC | (SEQ ID NO: 8) |
| ACAGATGTACTTATAAAGTGACTACATGCATTGCCTACTGCCTCGGA | |
| CND1K3: (19mer Sense Strand) GAATAGGCATTAACACAAA | (SEQ ID NO: 9) |
| CND1K3: (22mer Sense Strand) AAGAATAGGCATTAACACAAAG | (SEQ ID NO: 10) |
| TGCTGTTGACAGTGAGCGCAGAATAGGCATTAACACAAAGTAGTGAAGCC | |
| AC CND1K3: (shRNA) | |
| AGATGTACTTTGTGTTAATGCCTATTCTTTGCCTACTGCCTCGGA | (SEQ ID NO: 11) |
| CND1K4: (19mer Sense Strand) CATGAAAGTCTAGAAATAA | (SEQ ID NO: 12) |
| CND1K4: (22mer Sense Strand) AACATGAAAGTCTAGAAATAAA | (SEQ ID NO: 13) |
| CND1K4: (shRNA) | |
| TGCTGTTGACAGTGAGCGCACATGAAAGTCTAGAAATAAATAGTGAAGCC | (SEQ ID NO: 14) |
| ACAGATGTATTTATTTCTAGACTTTCATGTTTGCCTACTGCCTCGGA | |
| CND1K5: (19mer Sense Strand) AGGCCAGTATGATTTATAA | (SEQ ID NO: 15) |
| CND1K5: (22mer Sense Strand) TAAGGCCAGTATGATTTATAAA | (SEQ ID NO: 16) |
| CND1K5: (shRNA) | |
| TGCTGTTGACAGTGAGCGCAAGGCCAGTATGATTTATAAATAGTGAAGCC | (SEQ ID NO: 17) |
| ACAGATGTATTTATAAATCATACTGGCCTTATGCCTACTGCCTCGGA | |

| FGF19 Targeting Oligonucleotides Based on NM 005117 | |
|---|---|
| 19K1: (21mer Sense Strand) ACATGATATTTATGAGTAATT | (SEQ ID NO: 18) |
| 19K1: (22mer Sense Strand) AACATGATATTTATGAGTAATT | (SEQ ID NO: 19) |
| 19K1: (shRNA) | |
| TGCTGTTGACAGTGAGCGCACATGATATTTATGAGTAATTTAGTGAAGCC | (SEQ ID NO: 20) |
| ACAGATGTAAATTACTCATAAATATCATGTTTGCCTACTGCCTCGG A | |
| 19K2: (19mer Sense Strand) CAGGTGATCCACTCTGTAA | (SEQ ID NO: 21) |
| 19K2: (22mer Sense Strand) AACAGGTGATCCACTCTGTAAA | (SEQ ID NO: 22) |
| 19K2: (shRNA) | |
| TGCTGTTGACAGTGAGCGCACAGGTGATCCACTCTGTAAATAGTGAAGCC | (SEQ ID NO: 23) |
| ACAGATGTATTTACAGAGTGGATCACCTGTTTGCCTACTGCCTCGGA | |
| 19K3: (19mer Sense Strand) CCTCGAGGTTGCTGGACAA | (SEQ ID NO: 24) |
| 19K3: (22mer Sense Strand) TCCCTCGAGGTTGCTGGACAAG | (SEQ ID NO: 25) |
| 19K3: (shRNA) | |
| TGCTGTTGACAGTGAGCGCCCCTCGAGGTTGCTGGACAAGTAGTGAAGCC | (SEQ ID NO: 26) |
| ACAGATGTACTTGTCCAGCAACCTCGAGGGATGCCTACTGCCTCGG A | |
| 19K4: (19mer Sense Strand) CACGTTCTGTTTAGCTTTA | (SEQ ID NO: 27) |
| 19K4: (22mer Sense Strand) TCCACGTTCTGTTTAGCTTTAG | (SEQ ID NO: 28) |
| 19K4: (shRNA) | |
| TGCTGTTGACAGTGAGCGCCCACGTTCTGTTTAGCTTTAGTAGTGAAGCCA | (SEQ ID NO: 29) |
| CAGATGTACTAAAGCTAAACAGAACGTGGATGCCTACTGCCTCGGA | |
| 19K5: (21mer Sense Strand) AGCTGAAATTCTCTAATTTTT | (SEQ ID NO: 30) |
| 19K5: (22mer Sense Strand) AAGCTGAAATTCTCTAATTTTT | (SEQ ID NO: 31) |
| 19K5: (shRNA) | |
| TGCTGTTGACAGTGAGCGCAGCTGAAATTCTCTAATTTTTTAGTGAAGCCA | (SEQ ID NO: 32) |
| CAGATGTAAAAAATTAGAGAATTTCAGCTTTGCCTACTGCCTCGGA | |

Aptamers and Small Molecules

The present invention also provides therapeutic aptamers that specifically bind to FGF19 polypeptides, thereby modulating activity of the FGF19 polypeptide. An “aptamer” may be a nucleic acid molecule, such as RNA or DNA that is capable of binding to a specific molecule with high affinity and specificity (Ellington et al., Nature 346, 818-22 (1990); and Tuerk et al., Science 249, 505-10 (1990)). An aptamer will most typically have been obtained by in vitro selection for binding of a target molecule. For example, an aptamer that specifically binds a FGF19 polypeptide can be obtained by in vitro selection for binding to a FGF19 polypeptide from a pool of polynucleotides. However, in vivo selection of an aptamer is also possible. Aptamers have specific binding regions which are capable of forming complexes with an intended target molecule in an environment wherein other substances in the same environment are not complexed to the nucleic acid. The specificity of the binding is defined in terms of the comparative dissociation constants (Kd) of the aptamer for its ligand (e.g., a FGF19 polypeptide) as compared to the dissociation constant of the aptamer for other materials in the environment or unrelated molecules in general. A ligand (e.g., a FGF19 polypeptide) is one which binds to the aptamer with greater affinity than to unrelated material. Typically, the Kd for the aptamer with respect to its ligand will be at least about 10-fold less than the Kd for the aptamer with unrelated material or accompanying material in the environment. Even more preferably, the Kd will be at least about 50-fold less, more preferably at least about 100-fold less, and most preferably at least about 200-fold less. An aptamer will typically be between about 10 and about 300 nucleotides in length. More commonly, an aptamer will be between about 30 and about 100 nucleotides in length.

Methods for selecting aptamers specific for a target of interest are known in the art. For example, organic molecules, nucleotides, amino acids, polypeptides, target features on cell surfaces, ions, metals, salts, saccharides, have all been shown to be suitable for isolating aptamers that can specifically bind to the respective ligand. For instance, organic dyes such as Hoechst 33258 have been successfully used as target ligands for in vitro aptamer selections (Werstuck and Green, Science 282:296-298 (1998)). Other small organic molecules like dopamine, theophylline, sulforhodamine B, and cellobiose have also been used as ligands in the isolation of aptamers. Aptamers have also been isolated for antibiotics such as kanamycin A, lividomycin, tobramycin, neomycin B, viomycin, chloramphenicol and streptomycin. For a review of aptamers that recognize small molecules, see Famulok, Science 9:324-9 (1999).

An aptamer of the invention can be comprised entirely of RNA. In other embodiments of the invention, however, the aptamer can instead be comprised entirely of DNA, or partially of DNA, or partially of other nucleotide analogs. To specifically inhibit translation in vivo, RNA aptamers are preferred. Such RNA aptamers are preferably introduced into a cell as DNA that is transcribed into the RNA aptamer. Alternatively, an RNA aptamer itself can be introduced into a cell.

Aptamers are typically developed to bind particular ligands by employing known in vivo or in vitro (most typically, in vitro) selection techniques known as SELEX (Ellington et al., Nature 346, 818-22 (1990); and Tuerk et al., Science 249, 505-10 (1990)). Methods of making aptamers are also described in, for example, U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291.

Generally, in their most basic form, in vitro selection techniques for identifying aptamers involve first preparing a large pool of DNA molecules of the desired length that contain at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. The oligonucleotide pool is amplified using standard PCR techniques, although any means that will allow faithful, efficient amplification of selected nucleic acid sequences can be employed. The DNA pool is then in vitro transcribed to produce RNA transcripts. The RNA transcripts may then be subjected to affinity chromatography, although any protocol which will allow selection of nucleic acids based on their ability to bind specifically to another molecule (e.g., a protein or any target molecule) may be used. In the case of affinity chromatography, the transcripts are most typically passed through a column or contacted with magnetic beads or the like on which the target ligand has been immobilized. RNA molecules in the pool which bind to the ligand are retained on the column or bead, while nonbinding sequences are washed away. The RNA molecules which bind the ligand are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules which are capable of acting as aptamers for the target ligand. Once an aptamer sequence has been successfully identified, the aptamer may be further optimized by performing additional rounds of selection. For use in the present invention, the aptamer is preferably selected for ligand binding in the presence of salt concentrations and temperatures which mimic normal physiological conditions.

The unique nature of the in vitro selection process allows for the isolation of a suitable aptamer that binds a desired ligand despite a complete dearth of prior knowledge as to what type of structure might bind the desired ligand.

The association constant for the aptamer and associated ligand is preferably such that the ligand functions to bind to the aptamer and have the desired effect at the concentration of ligand obtained upon administration of the ligand. For in vivo use, for example, the association constant should be such that binding occurs well below the concentration of ligand that can be achieved in the serum or other tissue. Preferably, the required ligand concentration for in vivo use is also below that which could have undesired effects on the organism.

The present invention also provides small molecules and antibodies that specifically bind to a FGF19 polypeptide, thereby inhibiting the activity of a FGF19 polypeptide. Examples of small molecules include, without limitation, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes).

Antibodies

Another aspect of the invention pertains to antibodies. In some embodiments, an antibody that is specifically reactive with a FGF19 polypeptide may be used to detect the presence of a FGF19 polypeptide or to inhibit activity of a FGF19 polypeptide. In certain embodiments, one or more anti-FGF19 antibodies (e.g., antibodies that are specifically reactive with a FGF19 polypeptide) may be used in therapeutic applications described herein (e.g., to inhibit FGF19 activity).

In some embodiments, by using immunogens derived from a FGF19 peptide, anti-protein/anti-peptide antisera or monoclonal antibodies can be made by standard protocols (see, for example, Antibodies: A Laboratory Manual ed. by Harlow and Lane (Cold Spring Harbor Press: 1988)). A mammal, such as a mouse, a hamster or rabbit can be immunized with an immunogenic form of the FGF19 peptide, an antigenic fragment which is capable of eliciting an antibody response, or a fusion protein. In a particular embodiment, the inoculated mouse does not express endogenous FGF19, thus facilitating the isolation of antibodies that would otherwise be eliminated as anti-self antibodies. Techniques for conferring immunogenicity on a protein or peptide include conjugation to carriers or other techniques well known in the art. An immunogenic portion of a FGF19 peptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of a FGF19 polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a FGF19 polypeptide and monoclonal antibodies isolated from a culture comprising such hybridoma cells.

The term “antibody” as used herein is intended to include fragments thereof which are also specifically reactive with a FGF19 polypeptide. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a FGF19 polypeptide conferred by at least one CDR region of the antibody. In preferred embodiments, the antibody further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments, the invention makes available methods for generating novel antibodies that bind specifically to FGF19 polypeptides. For example, a method for generating a monoclonal antibody that binds specifically to a FGF19 polypeptide may comprise administering to a mouse an amount of an immunogenic composition comprising the FGF19 polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the FGF19 polypeptide. Once obtained, a hybridoma can be propagated in a cell culture, optionally in culture conditions where the hybridoma-derived cells produce the monoclonal antibody that binds specifically to the FGF19 polypeptide. The monoclonal antibody may be purified from the cell culture.

The term “specifically reactive with” as used in reference to an antibody is intended to mean, as is generally understood in the art, that the antibody is sufficiently selective between the antigen of interest (e.g., a FGF19 polypeptide) and other antigens that are not of interest that the antibody is useful for, at minimum, detecting the presence of the antigen of interest in a particular type of biological sample. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the Biacore binding assay, Bia-core AB, Uppsala, Sweden), sandwich assays (e.g., the paramagnetic bead system of IGEN International, Inc., Gaithersburg, Md.), western blots, immunoprecipitation assays, and immunohistochemistry.

### 3. Pharmaceutical Compositions

The methods and compositions described herein for treating a subject suffering from cancer may be used for the prophylactic treatment of individuals who have been diagnosed or predicted to be at risk for developing cancer. In this case, the composition is administered in an amount and dose that is sufficient to delay, slow, or prevent the onset of cancer or related symptoms. Alternatively, the methods and compositions described herein may be used for the therapeutic treatment of individuals who suffer from cancer. In this case, the composition is administered in an amount and dose that is sufficient to delay or slow the progression of the condition, totally or partially, or in an amount and dose that is sufficient to reverse the condition to the point of eliminating the disorder. It is understood that an effective amount of a composition for treating a subject who has been diagnosed or predicted to be at risk for developing cancer is a dose or amount that is in sufficient quantities to treat a subject or to treat the disorder itself.

In certain embodiments, compounds of the present invention (e.g., inhibitors of a FGF19 mRNA or protein) are formulated with a pharmaceutically acceptable carrier. For example, a FGF19 inhibitor can be administered alone or as a component of a pharmaceutical formulation (therapeutic composition). The subject compounds may be formulated for administration in any convenient way for use in human medicine.

In certain embodiments, the therapeutic methods of the invention include administering the composition topically, systemically, or locally. For example, therapeutic compositions of the invention may be formulated for administration by, for example, injection (e.g., intravenously, subcutaneously, or intramuscularly), inhalation or insufflation (either through the mouth or the nose) or oral, buccal, sublingual, transdermal, nasal, or parenteral administration. The compositions described herein may be formulated as part of an implant or device. When administered, the therapeutic composition for use in this invention is in a pyrogen-free, physiologically acceptable form. Further, the composition may be encapsulated or injected in a viscous form for delivery to the site where the target cells are present. Techniques and formulations generally may be found in Remington's Pharmaceutical Sciences, Meade Publishing Co., Easton, Pa. In addition to FGF19 inhibitors, therapeutically useful agents may optionally be included in any of the compositions as described above. Furthermore, therapeutically useful agents may, alternatively or additionally, be administered simultaneously or sequentially with FGF19 inhibitors according to the methods of the invention.

In certain embodiments, compositions of the invention can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Certain compositions disclosed herein may be administered topically, either to skin or to mucosal membranes. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the invention (e.g., a FGF19 inhibitor), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a subject compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

In certain embodiments, pharmaceutical compositions suitable for parenteral administration may comprise a FGF19 inhibitor in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

A person of ordinary skill in the art is able to determine the required amount to treat the subject. It is understood that the dosage regimen will be determined for an individual, taking into consideration, for example, various factors which modify the action of the subject compounds of the invention, the severity or stage of cancer, route of administration, and characteristics unique to the individual, such as age, weight, and size. A person of ordinary skill in the art is able to determine the required dosage to treat the subject. In one embodiment, the dosage can range from about 1.0 ng/kg to about 100 mg/kg body weight of the subject. The dose can be delivered continuously, or at periodic intervals. For example, on one or more separate occasions. Desired time intervals of multiple doses of a particular composition can be determined without undue experimentation by one skilled in the art. For example, the compound may be delivered hourly, daily, weekly, monthly, yearly (e.g., in a time release form) or as a one time delivery. As used herein, the term “subject” or “patient” means any individual animal capable of becoming afflicted with cancer. The subjects include, but are not limited to, human beings, primates, horses, birds, cows, pigs, dogs, cats, mice, rats, guinea pigs, ferrets, and rabbits. In some embodiments, the subject is a human being.

Samples used in the methods described herein (e.g., diagnostic, prognostic, therapeutic, etc., or any combination thereof) may comprise cells from the eye, ear, nose, teeth, tongue, epidermis, epithelium, blood, tears, saliva, mucus, urinary tract, urine, muscle, cartilage, skin, or any other tissue or bodily fluid from which sufficient DNA, RNA, protein, or other molecule or combinations of molecules can be obtained.

In some embodiments, the sample should be sufficiently processed to render the DNA, RNA, protein, etc., or any combination thereof that is present available for assaying in the methods described herein. For example, samples may be processed such that DNA from the sample is available for amplification or for hybridization to another polynucleotide. The processed samples may be crude lysates where available DNA or RNA is not purified from other cellular material. Alternatively, samples may be processed to isolate the available DNA, RNA, protein, etc., or any combination thereof from one or more contaminants that are present in its natural source. Samples may be processed by any means known in the art that renders DNA, RNA, protein, etc., available for assaying in the methods described herein. Methods for processing samples may include, without limitation, mechanical, chemical, or molecular means of lysing and/or purifying cells and cell lysates. Processing methods may include, for example, ion-exchange chromatography, size exclusion chromatography, affinity chromatography, hydrophobic interaction chromatography, gel filtration chromatography, ultrafiltration, electrophoresis, and immunoaffinity purification with antibodies specific for particular epitopes of the polypeptide.

### 4. Animal Models

In some embodiments, the invention features a transgenic mouse, whose genome comprises: an expression construct comprising an FGF19 coding sequence operably linked to an inducible promoter, and a genetic mutation that causes the transgenic mammal to have greater susceptibility to cancer than a mouse not comprising the genetic mutation, where expression of the FGF19 gene leads to formation of cancer in the transgenic mammal and the cancer regresses when expression of the FGF19 gene is reduced. Mutations that render the animal more susceptible to cancer include disabling mutations in a tumor suppressor gene and activating mutations in an oncogene. In a related embodiment, the induction of the FGF19 expression occurs in a tissue-specific manner, i.e., the FGF19 transgene can be turned on or off only in a particular tissue of the animal; this embodiment allows one to study the development (including maintenance), regression and recurrence of tumor in a selected tissue or organ (such as, for example, the liver or esophagus) of the animal as well as the efficacy and tissue toxicity of candidate drugs that target FGF19.

The invention further provides a chimeric mouse comprising the expression construct containing FGF19 nucleotide sequence; and a genetic mutation that causes said chimeric animal to have greater susceptibility to cancer than an animal not comprising said genetic mutation, wherein expression of the FGF19-coding nucleic acid leads to formation of cancer in said chimeric animal, and wherein said cancer regresses in said chimeric animal when expression of said FGF19-coding nucleic acid is reduced. In another embodiment, this invention provides the chimeric animal wherein both somatic and germ cells comprise the FGF19-coding nucleic acid.

This invention provides a chimeric mouse comprising a disruption of the endogenous FGF19 gene. In one embodiment, both copies of the endogenous FGF19 gene are disrupted. In another embodiment, the FGF19 gene is disrupted in a specific tissue. In yet another embodiment, the FGF19 gene is disrupted using RNAi. In a further embodiment, the RNAi is constitutive or inducible.

Tumors showing specific amplifications of candidate oncogenes in gene expression profiles can be outgrown in culture. Using stable RNAi, efficient knockdown of these genes can be achieved. Tumor cells with stable knockdown of a previously amplified gene can be re-transplanted into the mouse model of the current invention. Using this approach new therapeutic targets for cancer can be obtained and the specific consequences of knocking down an amplified gene with regard to tumor growth or metastases can be studied. Drug therapies that specifically inhibit the identified targets can be developed using the methods and compositions described herein.

The term over-expressed, over-expression, enhancement or increase in expression refers to an abundance of an expressed gene product that is higher than the abundance of that same product under other conditions or in other cells or tissues. Over-expression or increased expression may be effected, for example, by one or more structural changes to the gene's encoding nucleic acid or encoded polypeptide sequence (e.g., primary nucleotide or amino acid changes or post-transcriptional modifications such as phosphorylation), altered gene regulation (e.g., in the promoters, regulators, repressors or chromatin structure of the gene), a chemical modification, an altered association with itself or another cellular component, an altered subcellular localization, a modification which causes higher levels of activity through association with other molecules in the cell (e.g., attachment of a targeting domain) and the like.

The term inhibition, under-expressed, under-expression, inhibition or decrease in expression refers to an abundance of an expressed gene product that is lower than the abundance of the same product under other conditions or in other cells or tissues. Such under-expression or decreased expression may be effected, for example, by one or more structural changes to the gene's encoding nucleic acid or polypeptide sequence (e.g., primary nucleotide or amino acid changes or post-transcriptional modifications such as phosphorylation), altered gene regulation (e.g., in the promoters, regulators, repressors or chromatin structure of the gene), an altered structure (which causes reduced levels of activity), an altered association with itself or another cellular component, an altered subcellular localization, a modification which causes reduced levels of activity through association with other molecules in the cell (e.g., binding proteins which inhibit activity or sequestration) and the like.

The size and growth of cancer after therapy can be monitored by a wide variety of ways known in the art. Whole body fluorescence imaging can be used in the animal models of the invention, where the preferred viral vectors of this invention carry a GFP expression cassette. See, e.g., Schmitt et al., 2002, Cancer Cell 1:289-98. Tumors can also be examined histologically. Paraffin embedded tumor sections can be used to perform immunohistochemistry for cytokeratins and ki-67 as well as TUNEL-staining. The apoptotic rate of cells can be analyzed by TUNEL assay according to published protocols (Di Cristofano et al., 2001, Nature Genetics, 27:222-224). A significant regression or inhibition of the cancer in the mouse will indicate that the candidate molecule is useful for treating cancer.

### 5. Kits

Also provided herein are kits, e.g., kits for therapeutic purposes or diagnostic kits for detecting a amplified FGF19 gene in a sample from a patient. In one embodiment, a kit comprises at least one container means having disposed therein a polynucleotide probe that hybridizes, under stringent conditions, to the FGF19 gene. In another embodiment, a kit comprises at least one container means having disposed therein a polynucleotide primer that hybridizes, under stringent conditions, adjacent to one side of the FGF19 gene. In a further embodiment, a second polynucleotide primer that hybridizes, under stringent conditions, to the other side of the FGF19 gene is provided. In another embodiment, a kit comprises at least one container means comprising a premeasured dose of one or more inhibitors of FGF19. Kits further comprise a label and/or instructions for the use of the therapeutic or diagnostic kit in the detection of amplification of the FGF19 gene in a sample. Kits may also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber (see products available from www.papermart.com. for examples of packaging material).

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, Cold Spring Harbor Laboratory (2001); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Throughout this specification, the word “comprise” or variations such as “comprises” or “comprising” will be understood to imply the inclusion of a stated integer or group of integers but not the exclusion of any other integer or group of integers.

The following examples are meant to illustrate the methods and materials of the present invention. Suitable modifications and adaptations of the described conditions and parameters normally encountered in the art are within the spirit and scope of the present invention.

### Example 1

### Identification of FGF19 as a Driver Gene for the 11q13 Amplicon in Liver and Esophageal Cancers

The 11q13 amplicon containing the cyclin D1 gene is one of the most if not the most frequently amplified region in many types of cancers—up to 50% of oral carcinomas and 25% of esophageal tumors (Jiang, W., S. M. Kahn, et al. (1992). “Amplification and expression of the human cyclin D gene in esophageal cancer.” Cancer Res 52(10): 2980-3; Huang, X., T. E. Godfrey, et al. (2006). “Comprehensive genome and transcriptome analysis of the 11q13 amplicon in human oral cancer and synteny to the 7F5 amplicon in murine oral carcinoma.” Genes Chromosomes Cancer 45(11): 1058-69), 15% in both breast and liver cancer and from 5% to 30% in lung cancer Zhang, Y. J., W. Jiang, et al. (1993). “Amplification and over-expression of cyclin D1 in human hepatocellular carcinoma.” Biochem Biophys Res Commun 196(2): 1010-6; Ormandy, C. J., E. A. Musgrove, et al. (2003). “Cyclin D1, EMS1 and 11q13 amplification in breast cancer.” Breast Cancer Res Treat 78(3): 323-35; Gautschi, O., D. Ratschiller, et al. (2007). “Cyclin D1 in non-small cell lung cancer: a key driver of malignant transformation.” Lung Cancer 55(1): 1-14). Other regions near the CCND1 gene are often co-amplified or amplified independently, particularly in breast cancer, and appear to contain independent driver genes such as EMS1 (cortactin), but a core region around CCND1 is the most frequently amplified 11q13 region (Ormandy, C. J., E. A. Musgrove, et al. (2003). “Cyclin D1, EMS1 and 11q13 amplification in breast cancer.” Breast Cancer Res Treat 78(3): 323-35). Often the CCND1 core region is large and contains many genes, but the smallest amplicons observed encompass only a 280 kb region containing three genes, CCND1, ORAOV1/TAOS1, and FGF19 (chr11:69,047,008-69,274,977 Human May 2004 assembly). Previous analysis of expression and oncogenic function by several laboratories has established that CCND1 is a driver gene of this amplicon (Ormandy, C. J., E. A. Musgrove, et al. (2003). “Cyclin D1, EMS1 and 11q13 amplification in breast cancer.” Breast Cancer Res Treat 78(3): 323-35). ORAOV1/TAOS1 is over-expressed in oral cancers harboring 11q13 amplicons but its functional relevance has not been reported (Huang, X., S. M. Gollin, et al. (2002). “High-resolution mapping of the 11q13 amplicon and identification of a gene, TAOS1, that is amplified and over-expressed in oral cancer cells.” Proc Natl Acad Sci USA 99(17): 11369-74). FGF19 has been discounted based on lack of expression in oral cancer (Huang, X., T. E. Godfrey, et al. (2006). “Comprehensive genome and transcriptome analysis of the 11q13 amplicon in human oral cancer and synteny to the 7F5 amplicon in murine oral carcinoma.” Genes Chromosomes Cancer 45(11): 1058-69).

FGF19 was identified as a driver gene for the 11q13 amplicon in liver cancer, but not lung, melanoma or breast cancer, based on the correlation of DNA amplification and RNA expression in four different tumor types. Genome-wide profiling of expression was performed with Nimblegen expression arrays and of DNA copy number with 85K ROMA arrays. The Pearson correlation coefficients for FGF19 were 0.01 for breast, 0.76 for liver, −0.05 for lung, and −0.21 for melanoma. The correlation coefficient for liver was particularly high. The correlations for CCND1 were 0.55 for breast, −0.07 for liver, 0.12 for lung, and 0.14 for melanoma. Thus in liver, there was a better correlation of 11q13 DNA amplification with FGF19 expression then there was with CCND1.

### Example 2

### FGF19 RNA Over-Expression in Liver and Esophageal Cancer

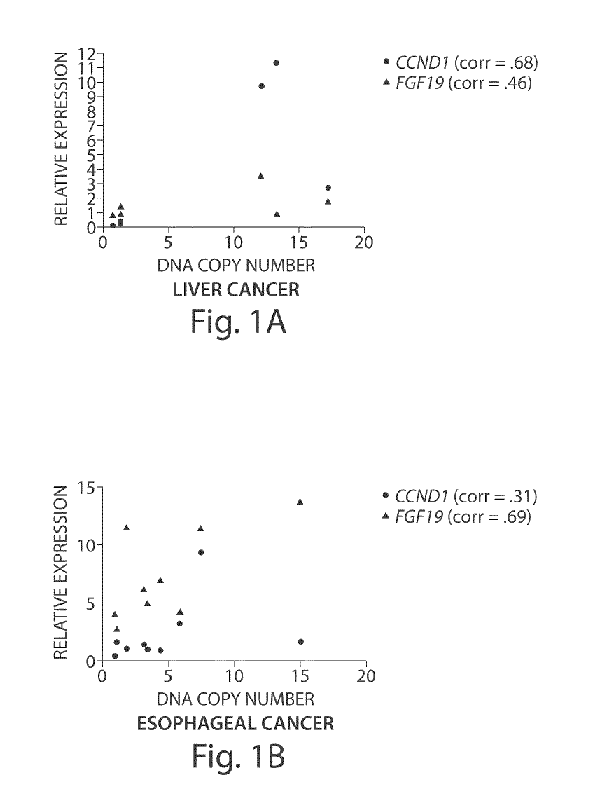

This prompted us to test by independent methods the correlation of DNA amplification and RNA expression in liver cancer (FIG. 1a) as well as esophageal cancer (FIG. 1b), a tissue which shares a common lineage with liver hepatocytes. Real time PCR (TaqMan) was used to determine whether FGF19 was over-expressed as a result of DNA amplification in these two tumor types. In both tumor types, both CCND1 and FGF19 are over-expressed as a result of DNA amplification (Liver tumors: CCND1 (corr=0.68); FGF19 (corr=0.46); Esophageal tumors: CCND1 (corr=0.31); FGF19 (corr=0.69)).

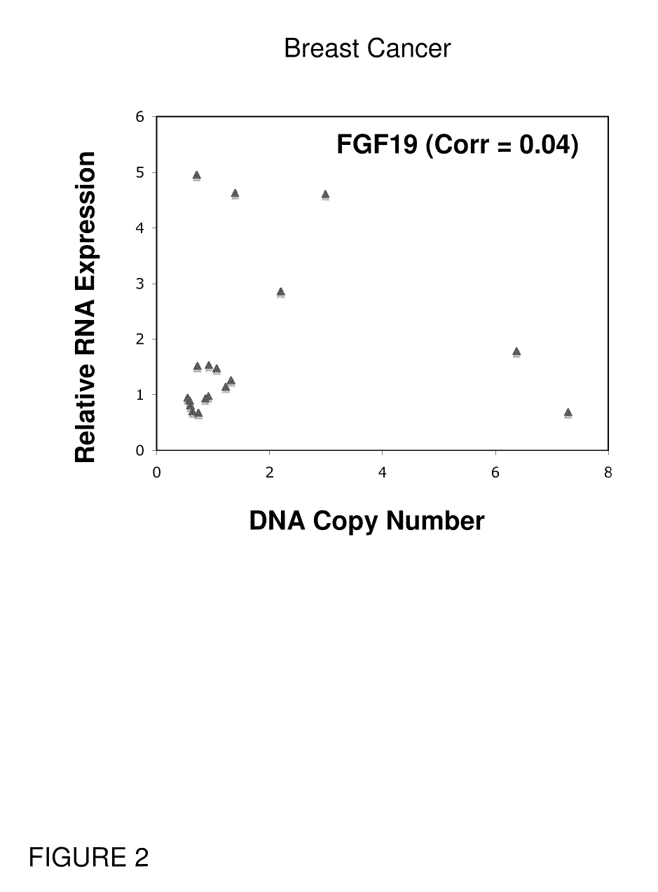

Using whole genome array analysis of both RNA expression and DNA copy number FGF19 was found not to be over-expressed as a result of DNA amplification in breast, lung, and melanoma cancers, but was over-expressed as a result of DNA amplification in liver cancer. (Table 1; See also FIG. 2 showing real time PCR (TaqMan) analysis indicating that FGF19 is over-expressed as a result of DNA amplification in Breast cancer). TaqMan analysis methods for DNA copy number and RNA expression are described, for example, in Zender L, et al., Identification and Validation of Oncogenes in Liver Cancer Using an Integrative Oncogenomic Approach, Cell 125, 1253-1267, Jun. 30, 2006. It should be appreciated that appropriate PCT techniques (e.g., RT-PCR, for example using TaqMan) may be used in any methods described herein (e.g., to detect increased copy number, to detect expression levels, for example over-expression or under-expression).

Surprisingly, in some embodiments, detecting amplification of 11q13 in human patients can serve as a predictor of response to therapies that block FGF19. There is evidence from two genes (HER2, MET) that amplification of oncogenes can serve as a predictor of response to inhibition of that same oncogene (Seidman, A. D., M. N. Fornier, et al. (2001). “Weekly trastuzumab and paclitaxel therapy for metastatic breast cancer with analysis of efficacy by HER2 immunophenotype and gene amplification.” J Clin Oncol 19(10): 2587-95; Smolen, G. A., R. Sordella, et al. (2006). “Amplification of MET may identify a subset of cancers with extreme sensitivity to the selective tyrosine kinase inhibitor PHA-665752.” Proc Natl Acad Sci USA 103(7): 2316-21).

TABLE 1
Whole genome array analysis of both RNA expression
and DNA copy number for >50 tumor samples per type
CORRELATION COEFFICIENT

| GENE | Breast | Lung | Liver | Melanoma |
|---|---|---|---|---|
| CCND1 | 0.54 | 0.17 | 0.65 | 0.10 |
| ORAOV1 | 0.79 | 0.57 | 0.82 | 0.69 |
| FGF19 | 0.01 | −0.09 | 0.67 | 0.05 |

### Example 3

TABLE 2
Properties of the Panel of Six Human
Hepatocellular Carcinoma Cell Lines

| | | | | Inhibition |
|---|---|---|---|---|
| | | aCGM | Detection of | of Colony |
| Liver Cancer | FGF19 | Segmented | Fgf19 | Formation |
| Cell Line | Amplication | Value | Protein | by RNAI |
| Huh 7 | + | 1.90 | + | + |
| Li7 | + | 4.03 | + | + |
| Hep3B | + | 2.58 | + | + |
| HepG2 | − | 0.96 | + | − |
| SNU182 | − | 0.99 | − | − |
| SNU423 | − | 1.02 | − | − |

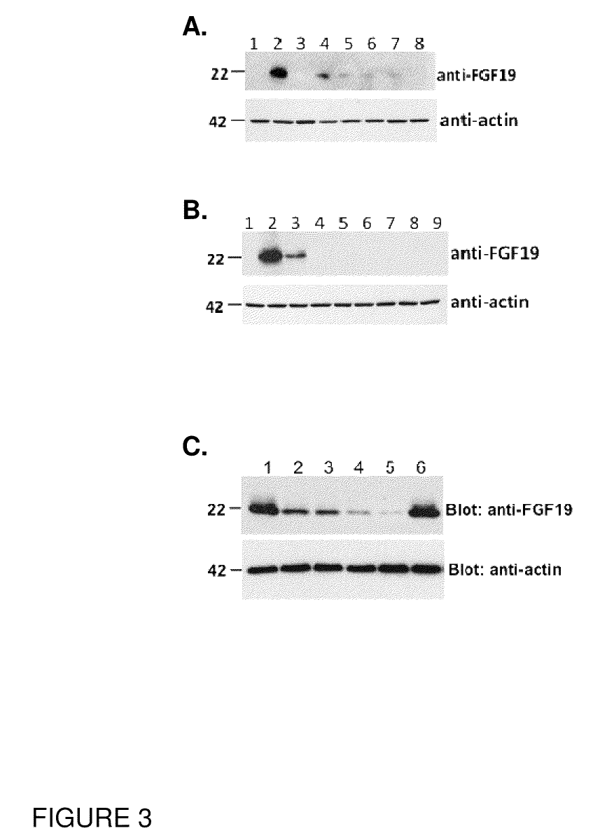

A panel of six human hepatocellular carcinoma cell lines was examined. The cells lines were chosen to equally represent human liver tumors with amplified FGF19 and CCND1 genes and non-amplified tumors. Relative DNA-copy number at the FGF19-CCND1 locus was determined by array CGH analysis using ROMA [1]. As shown in Table 2, the cell lines Huh7, Li7, and Hep3B all contain increased copy number of the FGF19 gene, whereas HepG2, SNU182, and SNU423 contain normal diploid levels. The level of Fgf19 protein also was measured in these various cell lines by immunoblotting with a commercially available antibody (R & D Biosystems) [FIG. 3]. As summarized in Table 1, Fgf19 protein could be detected in all three of the amplified cell lines, as well as one single-copy expressor, HepG2. To test for the requirement of FGF19 and CCND1 for tumorigenic properties, different shRNAs were designed to target FGF19 and CCND1 using the BIOPRED program as described in Zender et al. [2] and cloned these shRNAs into a retroviral expression vector, MSCV/LTRmir30 (courtesy of Scott Lowe, Cold Spring Harbor Laboratory). For all constructs, those with sufficient knockdown were determined by immunoblotting. Validation of the four critical anti-FGF19 shRNAs is shown in FIG. 3C.

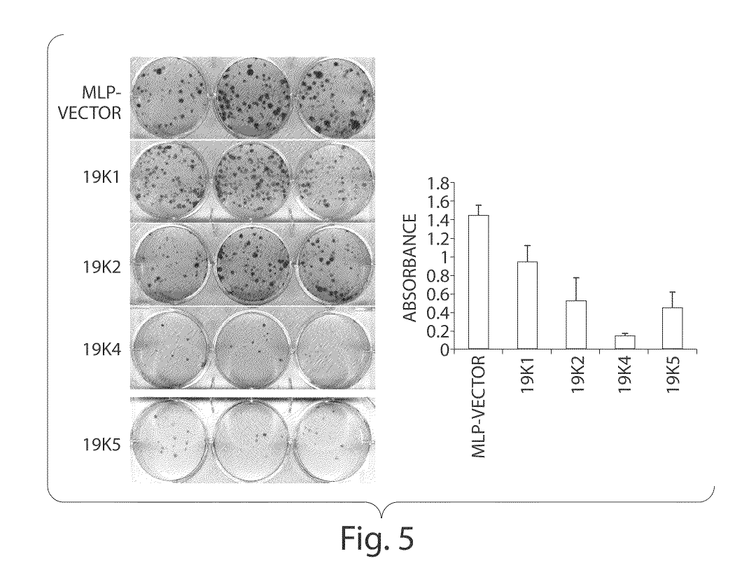

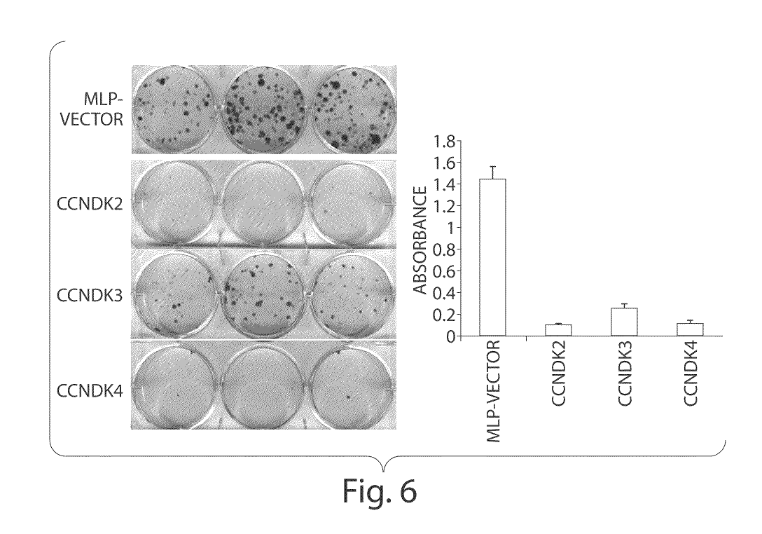

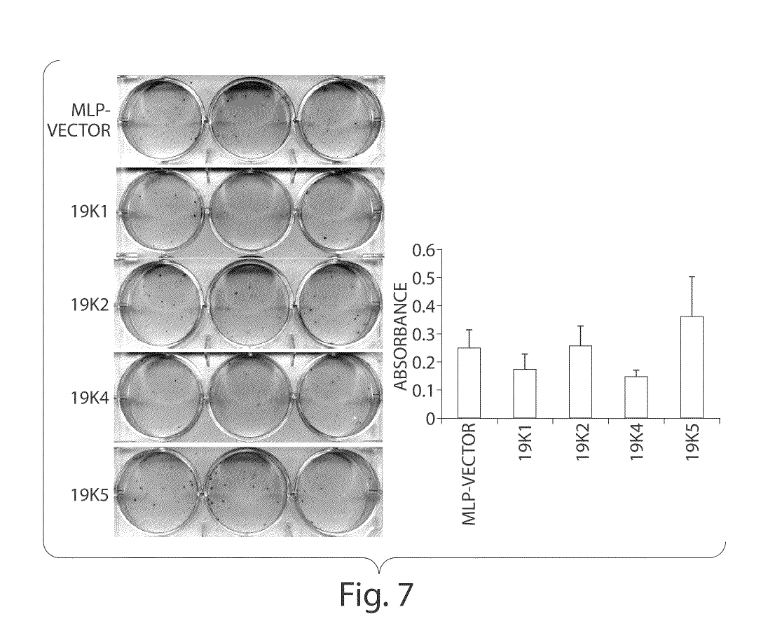

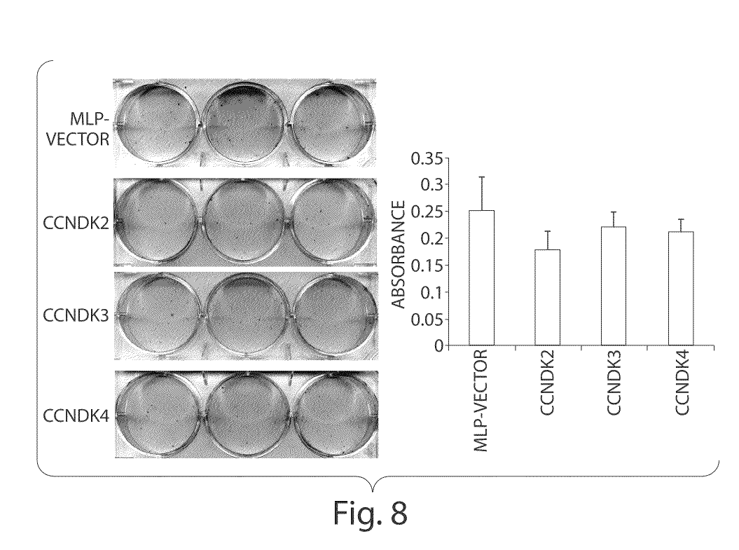

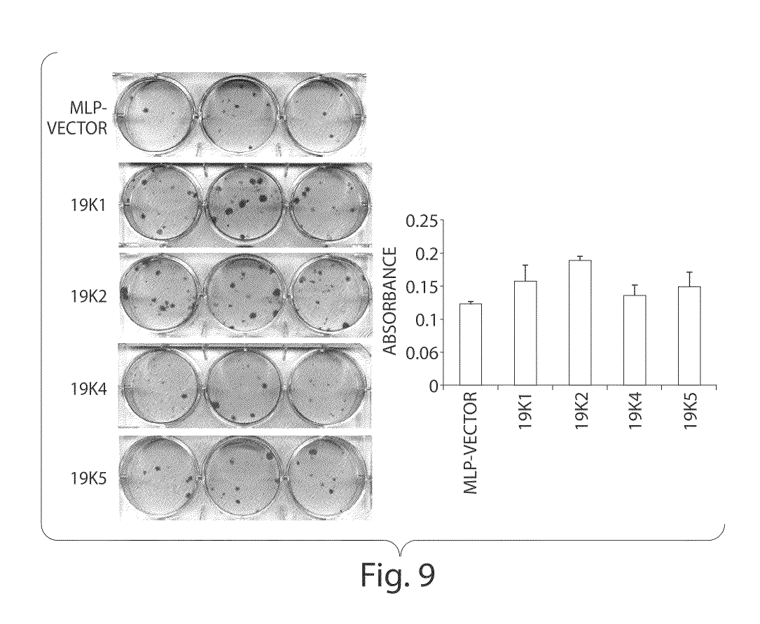

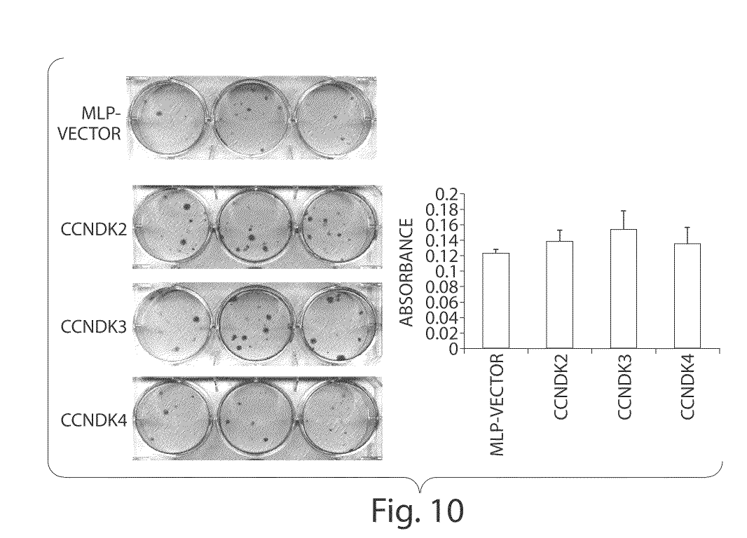

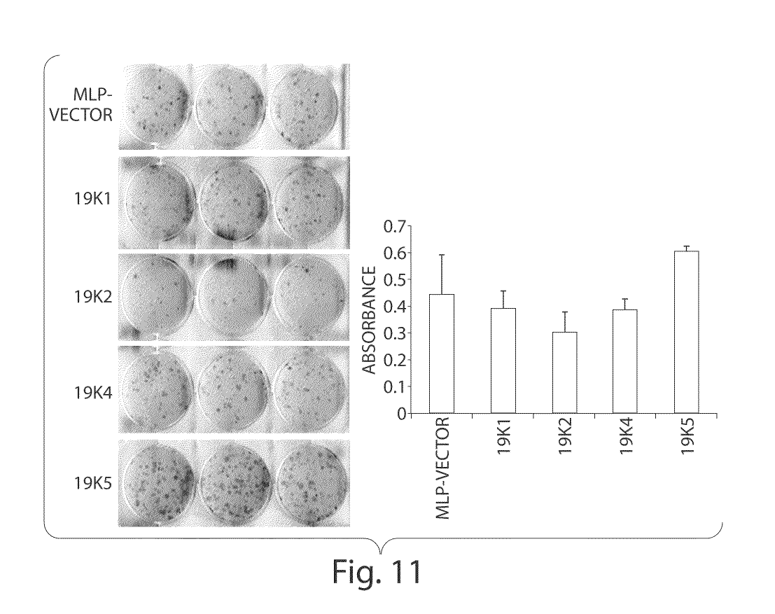

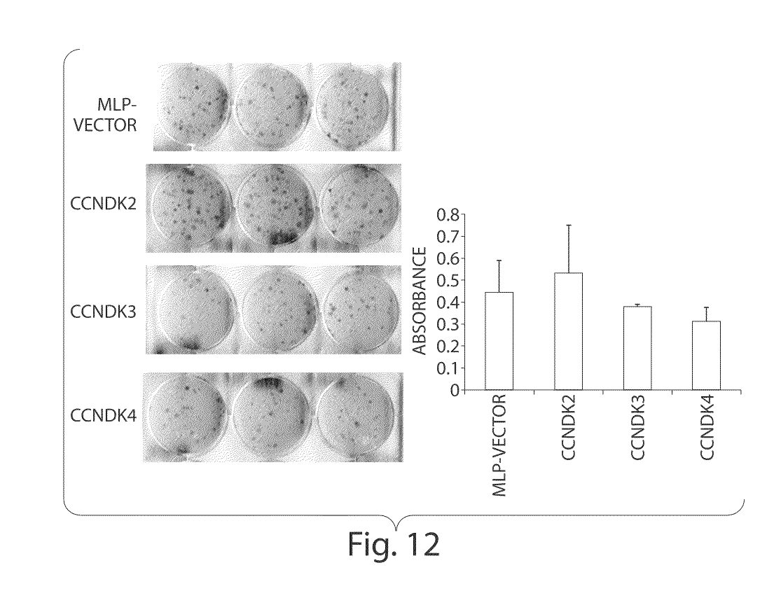

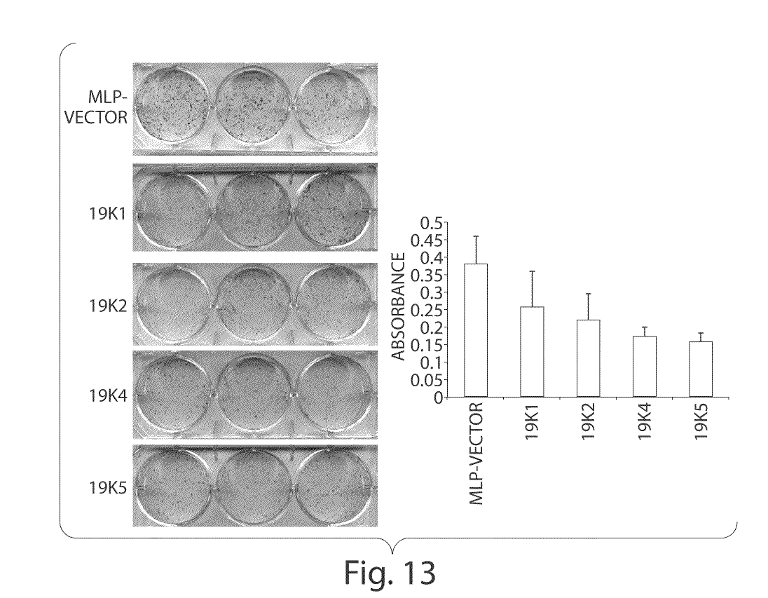

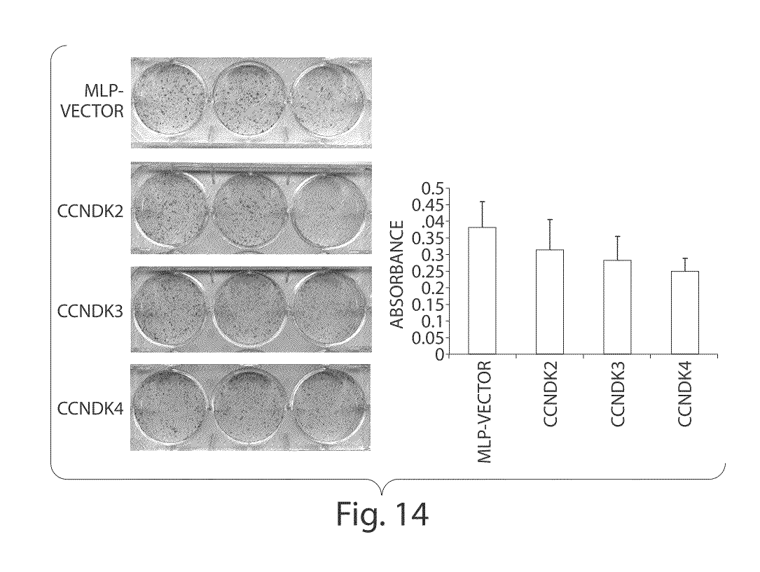

The effects of knocking down either FGF19 or CCND1 were then examined in this panel of six cancer cell lines, using an in vitro assay of clonogenicity that serves as a surrogate for tumorigenicity. Clonogenicity assays were performed by plating in triplicate in 6-well dishes at low density (depending on how fast the cells grew at higher density, e.g., plated at 500-1000 cells/well). After 2-3 weeks of growth, the cells were stained with crystal violet and dried overnight. The following day, pictures were taken (FIGS. 5-14) and the cells were then solubilized with Triton X-100 overnight in order to obtain quantitative absorbance readings. The summary of the results are shown in Table 2. Expression of Fgf19 protein alone did not correlate perfectly with sensitivity to inhibition of FGF19 by shRNAs, as the single copy expressor HepG2 was not affected by anti-FGF19 shRNAs. However, all three cell lines with amplification of FGF19 (and protein expression) were affected by anti-FGF19 shRNAs. The same three cell lines were specifically affected by anti-CCND1 shRNAs. Thus, amplification of the FGF19-CCND1 locus predicts response to inhibition of either FGF19 or CCND1.

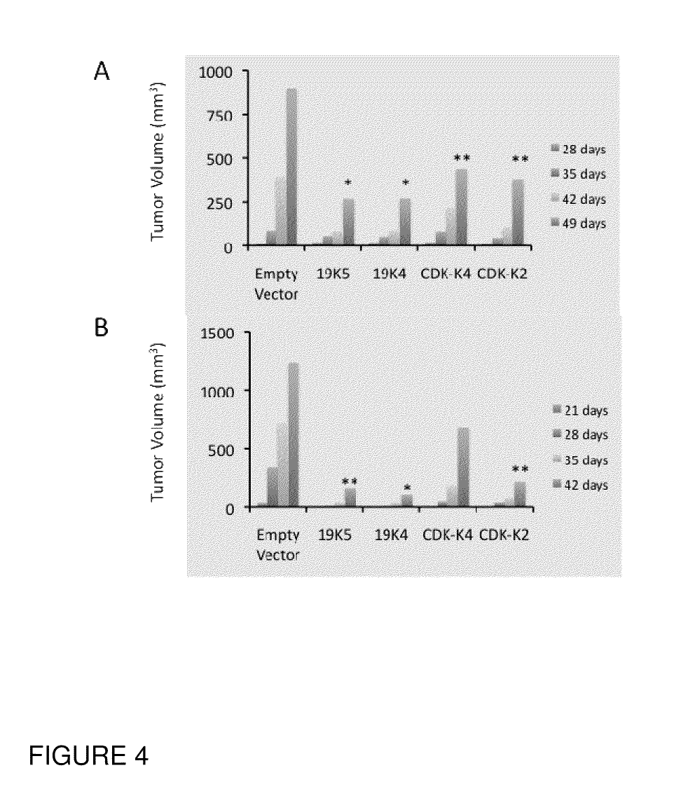

The effects of these shRNAs also were measured on tumor formation of the two amplified cell lines Huh7 and Li7. As shown in FIG. 4.

1. Lucito R, Healy J, Alexander J, Reiner A, Esposito D, Chi M, Rodgers L, Brady A, Sebat J, Troge J, et al.: Representational oligonucleotide microarray analysis: a high-resolution method to detect genome copy number variation. Genome Res 2003, 13:2291-2305.
2. Zender L, Xue W, Zuber J, Semighini C P, Krasnitz A, Ma B, Zender P, Kubicka S, Luk J M, Schirmacher P, et al.: An oncogenomics-based in vivo RNAi screen identifies tumor suppressors in liver cancer. Cell 2008, 135:852-864.

It will be clear that the invention may be practiced other than as particularly described in the foregoing description and examples. In some embodiments, methods of the invention include modifying, assaying, and/or optimizing one or more of the targeting or activity elements and/or the linker. Numerous modifications and variations of the present invention are possible in light of the above teachings and, therefore, are within the scope of the claims. Preferred features of each aspect of the invention are as for each of the other aspects mutatis mutandis. The documents including patents, patent applications, journal articles, abstracts, laboratory manuals, books, or other disclosures mentioned herein are hereby incorporated by reference in their entirety. In the event of conflict, the disclosure of present application controls, other than in the event of clear error. Further, the hard copy of the sequence listing submitted herewith and the corresponding computer readable form are both incorporated herein by reference in their entireties.

1. A method of identifying a patient as a candidate for treatment with an FGF19 inhibitor, the method comprising:
obtaining a clinical sample from a patient having cancer;
determining if a 11q13 locus is amplified in the clinical sample;
determining if FGF19 is over-expressed in the clinical sample; and,
if the clinical sample has amplification of the 11q13 locus and over-expression of FGF19, identifying the patient as a candidate for treatment with an FGF19 inhibitor.

2. A method of identifying a patient as a candidate for treatment with an FGF19 inhibitor, the method comprising:
obtaining a clinical sample from a patient having liver or esophageal cancer;
determining if a 11q13 locus is amplified in the clinical sample; and,
if the clinical sample has amplification of the 11q13 locus, identifying the patient as a candidate for treatment with an FGF19 inhibitor.

3. A method of identifying a patient as a candidate for treatment with an FGF19 inhibitor, the method comprising:
obtaining a clinical sample that is suspected of having an amplification of a 11q13 locus from a patient having cancer;
determining if FGF19 is over-expressed in the clinical sample; and,
if the clinical sample has over-expression of FGF19, identifying the patient as a candidate for treatment with an FGF19 inhibitor.

4. The method of claim 1, wherein the cancer is liver cancer or esophageal cancer.

5. The method of claim 1, wherein the step of determining if a 11q13 locus is amplified comprises:
measuring copy number of a 11q13 locus in the clinical sample.

6. The method of claim 1, wherein the step of determining if a 11q13 locus is amplified comprises:
comparing a copy number of the 11q13 locus in the clinical sample with a control sample, wherein an increase in copy number of the 11q13 locus compared with the control sample indicates that the 11q13 locus is amplified.

7. The method of claim 1, wherein the step of determining if a 11q13 locus is amplified comprises:
measuring copy number of FGF19 in the clinical sample.

8. The method of claim 1, wherein the step of determining if a 11q13 locus is amplified comprises:
comparing a copy number of FGF19 in the clinical sample with a control sample, wherein an increase in copy number of FGF19 compared with the control sample indicates that the 11q13 locus is amplified.

9. The method of claim 1, wherein the step of determining if a 11q13 locus is amplified comprises:
combining the clinical sample with a polynucleotide probe that hybridizes, under stringent conditions, to the 11q13 locus;
detecting hybridization of the polynucleotide probe; and
comparing the amount of hybridization that occurs in the clinical sample to the amount of hybridization that occurs in a control sample comprising a reference tissue, wherein an increased level of hybridization in the clinical sample relative to the control sample indicates that the 11q13 locus is amplified.

10. The method of claim 1, wherein the step of determining if a 11q13 locus is amplified comprises:
combining the clinical sample with a first polynucleotide probe that hybridizes, under stringent conditions, to the 11q13 locus;
combining the clinical sample with a second polynucleotide probe that hybridizes, under stringent conditions, adjacent to the first probe;
ligating the first and second polynucleotide probes to form a target probe;
amplifying the target probe; and
detecting the amount of amplified target probe, wherein an increased level of target probe in the clinical sample relative to a control sample comprising a reference tissue indicates that the 11q13 locus is amplified.

11. The method of claim 1, wherein the step of determining if a 11q13 locus is amplified comprises:
combining the clinical sample with a pair of polynucleotide primers that hybridize, under stringent conditions, to the 11q13 locus;
amplifying DNA in the sample, thereby producing amplified DNA; and
detecting the amount of amplified DNA, wherein an increased level of amplified DNA in the clinical sample relative to a control sample comprising a reference tissue indicates that the 11q13 locus is amplified.

12. The method of claim 9, wherein the polynucleotide probe comprises a sequence complementary to a FGF19 genomic sequence.

13. The method of claim 9, wherein the polynucleotide probe is a FISH probe, a Southern blot probe, a real-time PCR probe, array probe or a bead array probe.

14. The method of claim 10, wherein the target probe comprises a sequence complementary to a FGF19 genomic sequence.

15. The method of claim 11, wherein the polynucleotide primers comprise a sequence complementary to a FGF19 genomic sequence.

16. The method of claim 1, wherein the step of determining if FGF19 is over-expressed comprises:
measuring expression of FGF19 mRNA in the clinical sample.

17. The method of claim 1, wherein the step of determining if FGF19 is over-expressed comprises:
comparing expression of FGF19 mRNA in the clinical sample with a control sample, wherein an increase in expression of FGF19 mRNA in the clinical sample compared with the control sample indicates that FGF19 is over-expressed.

18. The method of claim 16, wherein the measuring comprises:
performing real-time PCR, FISH, northern analysis, a RNAse protection assay, microarray analysis, or bead array analysis to detect FGF19 mRNA.

19. The method of claim 1, wherein the step of determining if
FGF19 is over-expressed comprises:
measuring expression of FGF19 protein in the clinical sample.

20. The method of claim 1, wherein the step of determining if FGF19 is over-expressed comprises:
comparing expression of FGF19 protein in the clinical sample with a control sample, wherein an increase in expression of FGF19 protein in the clinical sample compared with the control sample indicates that FGF19 is over-expressed.

21. The method of claim 19, wherein the measuring comprises:
performing an ELISA or Immunohistochemistry to detect FGF19 protein.

22. The method of claim 1, wherein the clinical sample is a tissue biopsy, optionally wherein the tissue is a liver or esophageal tissue.

23. The method of claim 1, wherein the FGF19 inhibitor is selected from the group consisting of: an anti-FGF19 antibody or an antigen-binding fragment thereof, an anti-FGF19 antisense molecule, and, an aptamer, siRNA or miRNA against FGF19.

24. The method of claim 22, wherein the anti-FGF19 antibody is a mouse anti-human FGF-19 monoclonal antibody 1A6 or an antibody comprising a variable region from a mouse anti-human FGF-19 monoclonal antibody 1A6.

25. A method of treating a patient having liver or esophageal cancer, wherein amplification of the 11q13 locus is detected in a clinical sample from the patient, comprising administering to the patient an effective amount of an FGF19 inhibitor.

26. A method of treating a patient having cancer, wherein amplification of the 11q13 locus and over-expression of FGF19 is detected in a clinical sample from the patient, comprising administering to the patient an effective amount of an FGF19 inhibitor.

27. (canceled)

28. (canceled)

29. (canceled)
